(19) United States
(12) Patent Application Publication
(10) Pub. No.: US 2024/0301138 A1
(43) Pub. Date: Sep. 12, 2024

(54) POLMERIC TRANSFECTION REAGENTS TO DELIVER NUCLEIC ACIDS FOR HOST CELL MODIFICATION

(71) Applicant: RJH BIOSCIENCES INC., Edmonton (CA)

(72) Inventors: Remant Badahur KC, Edmonton, AB (CA); Amarnath Praphakar RAJENDRAN, Edmonton, AB (CA); Hasan ULUDAG, Edmonton, AB (CA)

(73) Assignee: RJH BIOSCIENCES INC., Edmonton, AB (CA)

(21) Appl. No.: 18/272,784

(22) PCT Filed: Jan. 14, 2022
(86) PCT No.: PCT/CA2022/050051

§ 371 Date: Jul. 17, 2023

Related U.S. Application Data
(60) Provisional application No. 63/138,626, filed on Jan. 18, 2021.

(51) Int. Cl. C08G 73/02; A61K 9/51; A61K 31/713; A61K 35/17; A61K 48/00; A61P 35/00

(52) U.S. Cl. CPC C08G 73/02; A61K 9/5138; A61K 9/5169; A61K 31/713; A61K 35/17; A61K 48/0041; A61P 35/00

(57) ABSTRACT

Effective polymeric transfection reagents for delivery of nucleic acids and their complexes to modify host cells, particularly hematopoietic cells including myeloid and lymphoid cells, pharmaceutical compositions comprising same, and methods of preparing and using same are provided. An effective compound comprises a low molecular weight polymer and aliphatic lipid-thioester or lipid-ester groups. A nanoparticle comprises the compound complexed with a nucleic acid and/or an additive. A composition or pharmaceutical composition comprises the nanoparticle and a pharmaceutically acceptable carrier. A method of treating, preventing, or ameliorating a disease in a subject comprises administering to the subject an effective amount of the nanoparticle or the composition or pharmaceutical composition.

### FIELD OF THE INVENTION

The present invention relates to polymeric transfection reagents for delivery of nucleic acids and their complexes to modify host cells, particularly hematopoietic cells including myeloid and lymphoid cells, pharmaceutical compositions comprising same, and methods of preparing and using same.

### BACKGROUND OF THE INVENTION

Polynucleotides (i.e., nucleic acids) are large, anionic macromolecules which cannot enter cells on their own and hence cannot exert any biological effect in the absence of a carrier. The delivery of nucleic acids to cells may be accomplished by various physical or chemical methods. Physical methods may include for example, disruption of the cell membrane by a force (e.g., electric current or pressure) to create holes through which polynucleotides can penetrate the cell membrane [1]. However, this is usually a toxic process and damage may be induced in the cells, leading to cell death or undesirable effects. Chemical methods may involve use of transfection reagents such as lipid-based carriers (e.g., liposomes, lipid particles, solid nucleic acid lipid particles) and cationic molecules (e.g., peptides, oligomers or larger cationic macromolecules) [2, 3]. Lipids are hydrophobic and require organic solvents for processing. Exposure of cells during modification to such solvents is undesirable. Small polyamines (e.g., spermine and related compounds) and larger polycations (e.g., polyamino acids and polylysine) have been modified with lipids to improve transfection using various chemical methods.

Conventional chemical delivery methods can transfect wide varieties of cell lines but display severe toxic effects at the optimum concentration required to achieve effective transfection. In hard-to-transfect cells, a significant concern is obtaining a high enough transfection efficiency required for translation to clinical applications. Hard-to-transfect cells include, among others, primary cells from a human host that have a finite lifetime and may be attachment-dependent cells or suspension-growing hematopoietic cells comprising myeloid and lymphoid cells, generally found in blood or soft tissues intimate with interstitial fluids such as bone marrow, spleen, and lymph nodes. Such cells display significantly lower transfection using chemical methods [4].

The ability to transfect hematopoietic (i.e., myeloid and lymphoid) cells in particular with nucleic acids such as DNA and RNA is highly desirable. Transfections alter hematopoietic cells at the genetic level and modulate their activity for intended therapeutic and diagnostic activities in the body. Modification of T-cells, for example, has been actively pursued to modulate a suppressed or over-stimulated immune system. T-lymphocytes are essential for adaptive immunity as they acquire T-cell receptors (TCRs) in the thymus to recognize foreign antigens from infectious pathogens and tumor antigens [5-7]. Since the 1980's, ex-vivo expanded T-cells have been used for treatment of diseases such as melanoma, cytomegalovirus and HIV [8-10]. The initial deployment of T-cells required simply sorting and expansion of allogeneic or autologous lymphocytes for their reintroduction into patients. However, obtaining sufficient numbers of disease-specific T-cells was difficult as patients usually possess limited cells that are reactive against the specific target [5, 11]. Relying on naturally expressed TCRs requires tumor antigens to be presented by specific major histocompatibility complexes (MHC), which are usually down-regulated or dysfunctional in many tumors besides being very specific to each patient [5, 12,13].

Engineered T-cells have emerged to better control the effectiveness of T-cell therapies [11]. Two T-cell based therapies recently approved by the FDA [14], Yescarta™ and Kymirah™, are genetically modified cells which express Chimeric Antigen Receptors (CARs) against CD19, an antigen present throughout the B-cell lineage [15-19]. CARs are recombinant receptor constructs, non-existent in nature and independent of HLA presentation, which combine a single-chain variable-fragment (scFv) with specificity to a target of interest which is commonly derived from a mAb fused to a T-cell signaling moiety joined by a transmembrane domain responsible for starting the effector response [20]. Most advanced CARs include co-stimulatory domains (commonly CD28 or 4-1BB) for more robust therapeutic responses [21-25].

Beyond hematopoietic cells, host cells important for clinical applications include fibroblasts that can be modified with a variety of factors to allow differentiation into specific phenotypes, or with stem cell factors to reverse them into a ‘stem-cell like’ phenotype that are suitable for modification and treatment of various diseases; bone marrow stromal cells that can be modified with growth factors, cytokines and transcription factors to form various cell phenotypes such as cartilage and bone; umbilical-cord derived cells for modification and use in various genetic defects in a host; and differentiated tissue-specific cells such as hepatocytes that can serve as the basis of artificial tissues for life support [26].

Therapeutic cells have been primarily modified by viral gene transfer which enabled permanent gene insertion into the genome [27]. However, viral gene transfer has been associated with high risk of insertional mutagenesis, especially when vectors are inserted close to growth-control genes, leading to oncogenesis and other toxicities [28-30]. The production of viral vectors is laborious, with production times ranging from 2 weeks to 6 months and it is sometimes difficult to achieve consistency among different batches or sources of virus [27, 31-34].

One alternative to viral modification is membrane pore-inducing electroporation. Electroporation-modified CAR T-cells, for example, have been shown to persist in the peripheral blood for more than 3 weeks and transgene expression was greater than 50% [35]. However, some drawbacks of electroporation include non-specific toxicity on the cells due to excessive pore formation. Longer ex vivo expansion might be required to allow cells to recover from electroporation, since grafting modified hematopoietic cells in a preclinical model was improved with longer culture times [36]. This approach cannot be used for in situ modification of patient cells due to limited access to target sites to apply electroporation [36].

Another alternative to viral modification is synthetic chemical methods that offer increased delivery loads and ease of manufacturing [37]. To date, lipid [38] and polymeric [39, 40] systems have been used for generating CAR T-cells with targeting capacity inducing tumor regression in a mouse model [40]. The chemical methods include lipid carriers (e.g., liposomes, lipid particles, solid nucleic acid lipid particles) and cationic molecules (e.g., oligomers or larger cationic macromolecules), small polyamines (e.g., spermine and related compounds), and poly (amino acids) such as poly(lysine). The cationic molecules have been further modified with hydrophobic and lipid molecules to create derivatives with improved performance [4].

Cationic polymers can be modified with functional groups for better performance using linkers that are stable under physiological conditions. Alternatively, linkers that are sensitive to endogenous stimuli can be employed to create materials that respond to local stimuli. These compounds could undergo physicochemical changes as a result of cleavage of the linkage and disruption of supramolecular structure with endogenous stimuli [41]. Redox-sensitive disulfide (—S—S—) is a common cleavable group that is inserted into polymers to generate effective delivery systems. The motivation for this approach is a “thiol-disulfide exchange reaction” that occurs in reductive environments, such as inside cells, which has a glutathione (GSH) concentration of 1 to 11 mM vs. extracellular space with GSH concentration of 2 to 10 μM. This allows prompt release of the payload intracellularly [42,43], while not allowing any cargo release outside the cells. The thioester linkage (—CO—S—) could also serve as a cleavable linker and can undergo cleavage via hydrolysis, aminolysis, or thiol-thioester exchange [44, 45]. In addition, regular ester linkage (—CO—O—) is a linkage that could be degraded by hydrolysis or with esterase enzymes in physiological environment and can serve as an additional linker for release of molecules.

Polyethylenimine (PEI) is the leading cationic polymer explored in gene delivery due to its facile chemistry, high buffering capacity and high cationic charge density important for nucleic acid binding [46-48]. Transfection efficiency of this polymer is generally proportional to the molecular weight, but unacceptable cellular toxicity for high molecular weight PEI is problematic for its translation to clinical applications. Low molecular weight PEIs are relatively safe but are ineffective as transfection reagents.

### SUMMARY OF THE INVENTION

The present invention relates to polymeric transfection reagents for delivery of nucleic acids and their complexes to modify host cells, particularly hematopoietic cells including myeloid and lymphoid cells, pharmaceutical compositions comprising same, and methods of preparing and using same.

In one aspect, the invention comprises a compound comprising a polymer having a molecular weight ranging from about 0.5 kDa to about 5 kDa and an aliphatic lipid-thioester group, and having the formula IB:

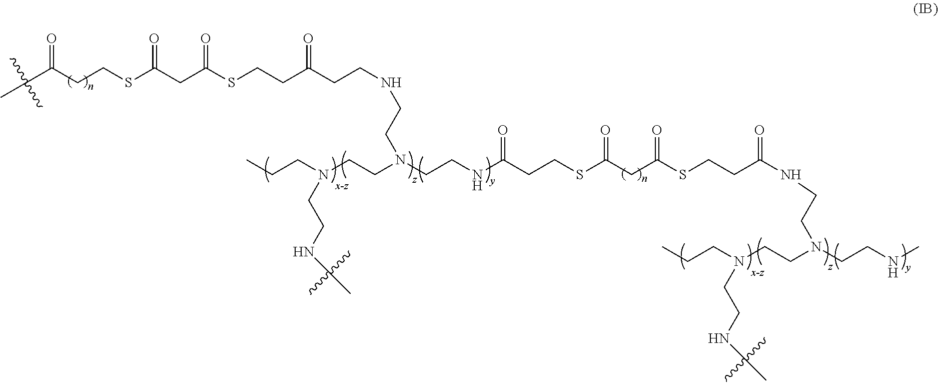

wherein the linker comprises a spacer of 3<n<22 atoms; x=5<n<30; y=5<n<30; and z=1<n<5.

In one embodiment, the aliphatic lipid-thioester group has the formula IIIA or IIIB:

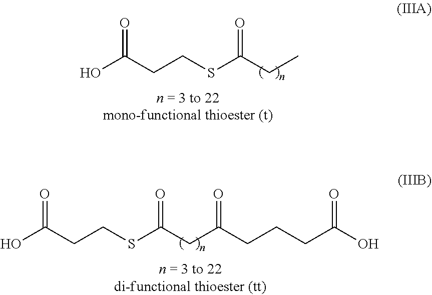

where n is the carbon chain length ranging from C3 to C22.

In one aspect, the invention comprises a compound comprising a polymer having a molecular weight ranging from about 0.5 kDa to about 5 kDa and a lipid-ester or lipid-thioester group, and having the formula IIA, IIB, IIC, or IID:

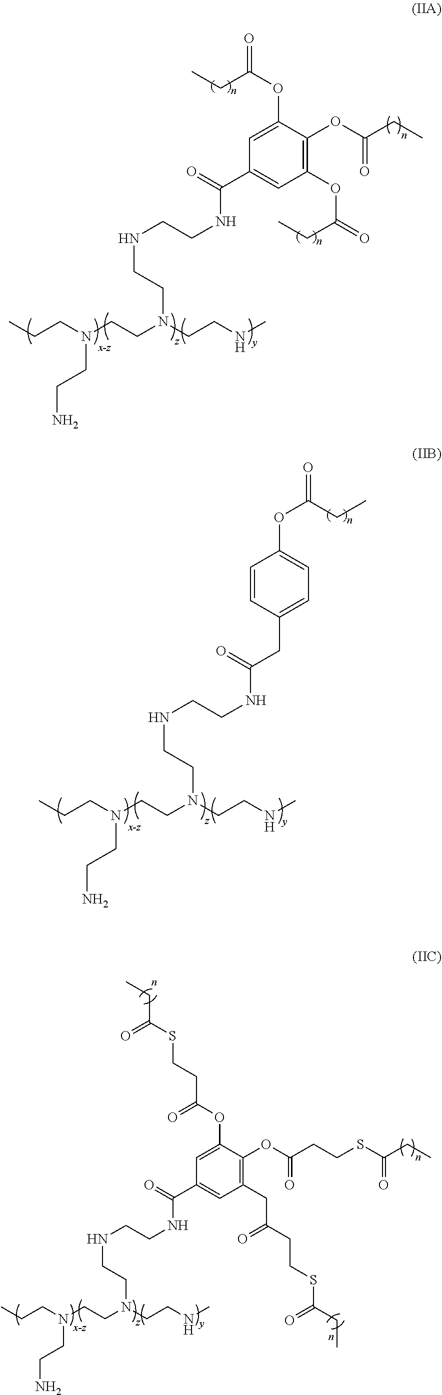

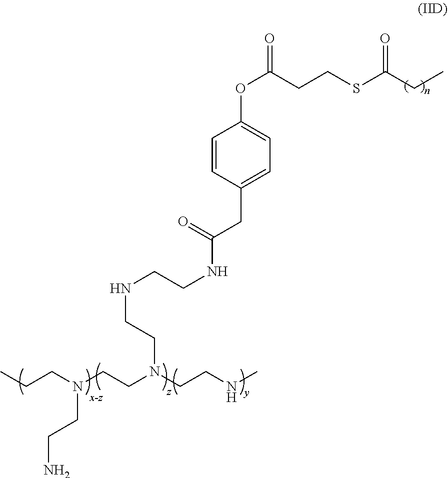

where the compound comprises a carbon chain length of 3<n<22 atoms; x=5<n<30; y=5<n<30; and z=1<n<5.

In one embodiment, the lipid-ester or lipid-thioester group has the formula IVA, IVB, IVC, or IVD:

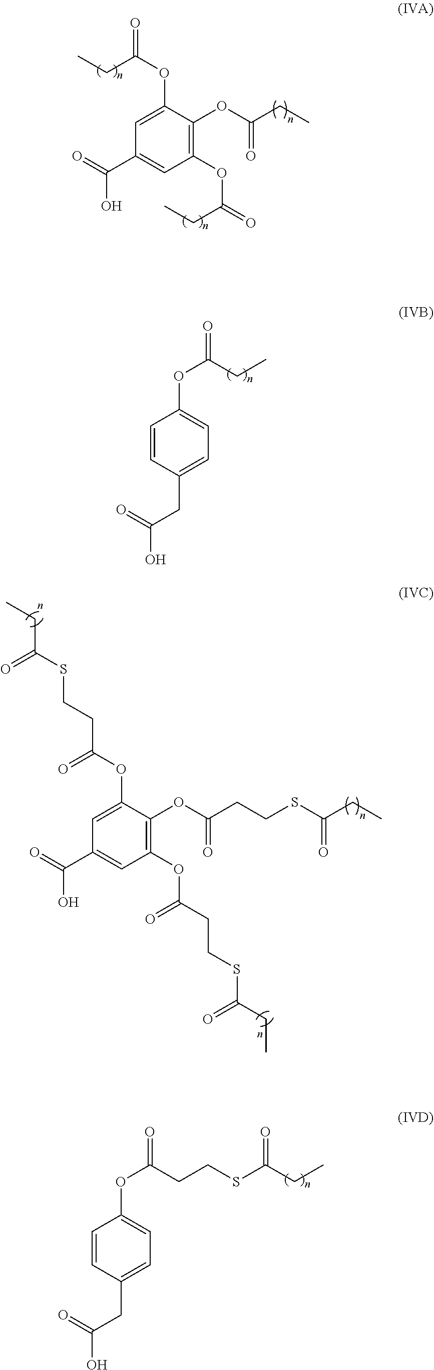

where n is the carbon chain length ranging from C3 to C22.

In one embodiment, the polymer is selected from polyethylenimine in a branched, linear, or dendritic form, polyalkylimine, a poly(amino acid), a poly(beta-amino acid), a poly(beta-amino ester), a cationic amino acid containing a peptide or a polymer, an aminated polymer derived from water-soluble, uncharged polymers modified with amine compounds, polyethylenimine derivatized with silica, polyethylenglycol, polypropyleneglycol, an amino acid, dopamine, poly(2-dimethylaminoethyl methacrylate or a derivative thereof in combination with a polymer to create amphiphilic polymers; a polyamidoamine derivative; and poly(N-(2-hydroxypropyl)methacrylamide) or a derivative thereof.

In one embodiment, the lipid comprises a saturated or unsaturated aliphatic lipid selected from propanoyl (C3), propanedioyl (C3), pentanedioyl or glutaryl chloride (C5), hexanoic acid or hexanoyl (C6), heptanedioyl or pimeloyl chloride (C7), capryloyl (C8), lipoic acid or lipoyl (C8), nonanedioyl or azelaoyl chloride (C9), lauric acid or lauroyl (C12), dodecanedioyl (C12), palmitic acid or palmitoyl (C16), stearoyl (C18), linoleoyl (C18), oleoyl (C18), eicosanoyl (C20), eicosapentaenoyl (C20), arachidonoyl (C20), linolarachidonoyl (C20), docosanoyl (22), docosahexaenoyl (22), myristoleic acid (C14:1, cis-9), palmitoleic acid (C16:1, cis-9), stearic acid (C18) and derivatives thereof, oleic acid (C18:1, cis-9), elaidic acid (C18:1, trans-9), linoleic acid (C18:2, cis-9,12), or linolenic acid (C18:3, cis-9,12,15); triglyceride including glyceryl tridecanoate, glyceryl tridodecanoate, glyceryl trimyristate, glyceryl trioctanoate, tripalmitin; lipoic acid and derivatives thereof in oxidized and reduced form; cholesterol and derivatives thereof including cholic acid, deoxycholic acid, and cholanic acid; phospholipid selected from α-phosphatidylcholine, α-phosphatidylethanolamine, α-phosphatidyl-L-serine, α-phosphatidylinositol, α-phosphatidic acid, α-phosphatidyl-DL-glycerol, α-lysophosphatidylcholine, sphingomyelin, cardiolipin; synthetic lipidic compounds including diphytanoyl phosphatidylethanolamine (DPHPE), dioleoyl phosphatidylethanolamine (DOPE), dioleoyl phosphatidylcholine (DOPC), dilauryl phosphatidylethanolamine (DLPE), 1,2-distearoyl-sn-glycero-3-phosphatidylethanolamine (DSPE), and dimyristoyl phosphatidylethanolamine (DPME); multicyclic lipids and steroids including cholesterol, cholestanol, coprosterol, epicholestanol, epicholesterol, ergostanol, [alpha]-ergostenol, [beta]-ergostenol, [gamma]-ergostenol, ergosterol, 22,23-dihydroergosterol, stigmasterol, stigmastanol, (3[beta])-7-dehydrocholesterol, desmosterol, allocholesterol, 24-hydroxycholesterol, 25-hydroxycholesterol, campesterol, [alpha]-sitosterol, [beta]-sitosterol, [gamma]-sitosterol, lumisterol, pyrocalciferol, isopyrocalciferol, azacosterol, neoergosterol, and dehydroergosterol.

In one aspect, the invention comprises a nanoparticle comprising the compound of formula IA, IB, IIA, IIB, IIC, or IID complexed to a nucleic acid. In one embodiment, the nucleic acid is selected from an RNA-based nucleic acid comprising siRNA, sgRNA, microRNA, mRNA, shRNA, or combinations thereof, a DNA-based nucleic acid comprising a DNA-based oligonucleotide or antisense oligonucleotide, plasmid DNA for encoding an RNA product comprising shRNA, mRNA, sgRNA, or combinations thereof, a peptide-nucleic acid; a DNA-RNA chimera; or a nucleic acid in combination with a protein. In one embodiment, the sgRNA is complexed to a DNA-editing enzyme comprising Cas9.

In one embodiment, the nanoparticle further comprises an additive selected from polyanions, polyacrylic acid, polymethacrylic acid, polyaspartic acid, polyglutamic acid, gelatin, hyaluronic acid, cellulose, or derivatives thereof.

In one aspect, the invention comprises a composition or pharmaceutical composition comprising a compound of formula IA, IB, IIA, IIB, IIC, or IID, or a nanoparticle comprising the compound of formula IA, IB, IIA, IIB, IIC, or IID complexed to a nucleic acid, and a pharmaceutically acceptable carrier.

In one aspect, the invention comprises a method of treating, preventing, or ameliorating a disease in a subject, comprising administering to the subject an effective amount of a nanoparticle comprising the compound of formula IA, IB, IIA, IIB, IIC, or IID complexed to a nucleic acid, or a composition or pharmaceutical composition comprising a compound of formula IA, IB, IIA, IIB, IIC, or IID, or a nanoparticle comprising the compound of formula IA, IB, IIA, IIB, IIC, or IID complexed to a nucleic acid, and a pharmaceutically acceptable carrier. Diseases include, but are not limited to, chronic and acute myeloid leukemia, chronic and acute lymphocytic leukemia, hairy cell leukemia, meningeal leukemia, myeloma, multiple myeloma, lymphoma, brain cancer, bladder cancer, breast cancer, melanoma, skin cancer, epidermal carcinoma, colon and rectal cancer, lung cancer, non-Hodgkin lymphoma, Hodgkin lymphoma, Sezary Syndrome, endometrial cancer, pancreatic cancer, kidney cancer, prostate cancer, leukemia thyroid cancer, head and neck cancer, ovarian cancer, hepatocellular cancer, cervical cancer, sarcoma, gastric cancer, gastrointestinal cancer, and uterine cancer.

In one embodiment, the disease is treated, prevented, or ameliorated in the subject through genetically modified hematopoietic host cells. In one embodiment, the host cells are selected from T-cells including helper T-cells, or Natural Killer cells. In one embodiment, the nanoparticle, composition or pharmaceutical composition comprises a nucleic acid selected from DNA or RNA.

In one aspect, the invention comprises use of a nanoparticle comprising the compound of formula IA, IB, IIA, IIB, IIC, or IID complexed to a nucleic acid, or a composition or pharmaceutical composition comprising a compound of formula TA, IB, IIA, IIB, IIC, or IID, or a nanoparticle comprising the compound of formula TA, IB, IIA, IIB, IIC, or IID complexed to a nucleic acid, and a pharmaceutically acceptable carrier, to treat, prevent, or ameliorate a disease in a subject.

In one aspect, the invention comprises a method of delivering mRNA or a ribonucleotide protein complex (RNP) using a compound having the formula IA, IB, IIA, IIB, IIC, or IID. In one embodiment, the compound has the formula IA:

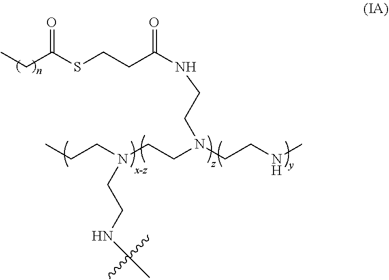

wherein the hydrophobic group comprises 3<n<22 atoms; x=5<n<30; y=5<n<30; and z=1<n<5.

Additional aspects and advantages of the present invention will be apparent in view of the description, which follows. It should be understood, however, that the detailed description and the specific examples, while indicating preferred embodiments of the invention, are given by way of illustration only, since various changes and modifications within the scope of the invention will become apparent to those skilled in the art from this detailed description.

### BRIEF DESCRIPTION OF THE DRAWINGS

The invention will now be described by way of an exemplary embodiment with reference to the accompanying simplified, diagrammatic, not-to-scale drawings. In the drawings:

FIG. 1A is a reaction scheme for preparing a mono-functional thioester (t) of formula (IIIA).

FIG. 1B is a reaction scheme for preparing a di-functional thioester (tt) of formula (IIIB).

FIG. 1C shows ¹H-NMR spectra of di-functional thioesters (tt) of formula (IIIB).

FIG. 1D shows mass spectroscopy spectra of di-functional thioesters (tt) of formula (IIIB).

FIG. 2A is a reaction scheme between a mono-functional thioester (t) of formula (IIIA) and PEI to yield a cleavable hydrophobic derivative of PEI (PEI-t) of formula (IA).

FIG. 2B is a reaction scheme between a di-functional thioester (tt) of formula (IIIB) and PEI to yield a cleavable crosslinked PEI (PEI-tt) of formula (IB).

FIG. 3A is a reaction scheme for preparation of a tri-functional ester of formula (IVA), and subsequent modification of PEI to yield an ester-linked lipopolymer of formula (IIA).

FIG. 3B is a reaction scheme for preparation of a mono-functional ester of formula (IVB), and subsequent modification of PEI to yield an ester-linked lipopolymer of formula (IIB).

FIG. 3C is a reaction scheme for preparation of a tri-functional ester of formula (IVC), and subsequent modification of PEI to yield a thioester-linked lipopolymer of formula (IIC).

FIG. 3D is a reaction scheme for preparation of a mono-functional ester of formula (IVD), and subsequent modification of PEI to yield a thioester-linked lipopolymer of formula (IID).

FIG. 3E shows ¹H-NMR spectra of ester-linked lipopolymers of formulae (IIA and IIB). GA refers to aromatic gallic acid linker used in formula (IIB) and PUPA refers to aromatic benzene linker used in formula (IIA).

FIG. 4A are graphs showing the DNA binding capacity (BC₅₀; polymer/DNA weight ratio at 50% plasmid DNA binding) of PEI-tt polymers as a function of C-chain length of crosslinker (i) and BC₅₀ of PEI-GA and PEI-PHPS polymers as a function of feed ratio during synthesis (ii).

FIG. 4B is a graph showing cytotoxicity in Jurkat cells transfected with crosslinked PEI-tt polymers as determined by the MTT Assay.

FIGS. 5A-B are graphs showing transgene expression in Jurkat cells with delivery of GFP-mRNA using PEI-tt polymers, as determined by flow cytometry analysis, which is summarized as the mean fluorescence intensity per cell (A) and GFP-positive cell population (B). FIGS. 5C-D are graphs showing transgene expression in Jurkat cells with the delivery of gWIZ-GFP using PEI-tt polymers, as determined by flow cytometry analysis, which is summarized as the mean fluorescence intensity per cell (C) and GFP-positive cell population (D).

FIGS. 6A-B are graphs showing transgene (GFP) expression in Jurkat cells by the delivery of GFP-mRNA (FIG. 6A) and gWIZ-GFP (FIG. 6B) using PEI-tt polymers, as determined by flow cytometry analysis, which is summarized as the mean fluorescence intensity per cell and GFP-positive cell population. The additive PAA was added to the formulations at a ratio of 0.5.

FIGS. 7A-B are graphs showing transgene (GFP) expression in Jurkat cells by delivery of GFP-mRNA (FIG. 7A) and gWIZ-GFP (FIG. 7B) using PEI-t polymers, as determined by flow cytometry, which is summarized as the mean fluorescence intensity per cell and GFP-positive cell population. The additive PAA was added to the formulations at a ratio of 0.5.

FIG. 8 shows images of transgene expression in Jurkat cells from fluorescent microscopy of a reporter Green Fluorescent Protein (GFP).

FIGS. 9A-E show images of transgene expression in kidney fibroblast cells transfected with crosslinked PEI-tt. FIGS. 9F-M are graphs of the transgene expression as determined by flow cytometry analysis, which is summarized as fluorescence micrographs, fluorescence intensity per cell and GFP-positive cell population.

FIGS. 10A-E show images of transgene expression in breast cancer MDA-MB-436 cells transfected with crosslinked PEI-tt. FIGS. 10F-M are graphs of the transgene expression as determined by flow cytometry analysis, which is summarized as the mean fluorescence intensity per cell and GFP-positive cell population.

FIGS. 11A-B are graphs of cell killing by human peripheral blood mononuclear cells (PBMCs) enriched for T-cells by culturing with IL-2. The cells were modified with a plasmid DNA and mRNA expression system for chimeric antigen receptors directed against human (FIG. 11A) and mouse (FIG. 11B) CD19. In FIG. 11A, the modified cells were incubated with human CD19+ RS4;11 cells. In FIG. 11B, the modified cells were incubated with mouse CD19+ WEHI cells.

FIGS. 12A-B are graphs showing siRNA delivery with modified PEIs (FIG. 12A) and treatment with remdesivir (FIG. 12B) to prevent cell death in Vero cells due to coronavirus infection, which is summarized as the percent cell viability as a function of siRNA or drug concentration.

FIGS. 13A-B are graphs showing delivery of ribonucleotide protein (RNP) complex comprising sgRNA and Cas9 enzyme using a modified PEI to edit MDA-MB-231 cells, which is summarized as the reduction in GFP fluorescence and in percentage of GFP-positive cells.

FIGS. 14A-B are graphs showing the effectiveness of the Polymer IA to deliver TRAIL mRNA using SUM-149 xenografts in mice.

### DETAILED DESCRIPTION OF PREFERRED EMBODIMENTS

Before the present invention is described in further detail, it is to be understood that the invention is not limited to the embodiments described, as such may, of course, vary. It is also to be understood that the terminology used herein is for the purpose of describing embodiments only, and is not intended to be limiting, since the scope of the present invention will be limited only by the appended claims. Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both limits, ranges excluding either or both of those included limits are also included in the invention. Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, a limited number of the exemplary methods and materials are described herein. It must be noted that as used herein and in the appended claims, the singular forms “a,” “an,” and “the” include plural referents unless the context clearly dictates otherwise.

The present invention relates to polymeric transfection reagents for delivery of nucleic acid and their complexes to modify host cells, particularly hematopoietic cells including myeloid and lymphoid cells, pharmaceutical compositions comprising same, and methods of preparing and using same. As used herein, the term “polymeric transfection reagent” generally refers to a polymer modified with hydrophobic and/or lipid groups, and which exhibits the ability to bind and deliver nucleic acid to a host cell, thereby modifying the host cell to confer a desired utility or to achieve a desired outcome. In one embodiment, the polymeric transfection reagents are responsive to local stimuli. In one embodiment, the response involves exhibiting degradation upon exposure to host factors.

In the development of the present invention, chemical modification of polymers involved either preparing crosslinked polymers or grafting lipid groups on polymers to yield transfection reagents for nucleic acid delivery. Since the efficacy and toxicity of polymers is proportional to their molecular weight, low molecular weight polymers, which are generally ineffective alone in their native state, require chemical modification. As used herein, the term “low molecular weight” means a molecular weight ranging from about 0.5 kDa to about 5 kDa, and more preferably from about 0.6 kDa to about 2.5 kDa. Low molecular weight polymers were modified to yield higher molecular weight polymers by either crosslinking with cleavable linkers, or grafting with lipids via specific chemical bonding. Without being bound by any theory, modification of polymers using lipid groups may enhance nanoparticle formation with nucleic acids and cellular affinity, and facilitate release of nucleic acids inside cells once internalized.

As will be described herein, the polymeric transfection reagents of the present invention generally comprise a polymer, a lipid, and a crosslinker. Suitable polymers include, but are not limited to, linear, branched, or dendritic forms of polyethylenimine (PEI) and other polyalkylimines including polypropylenimine; linear, branched, or dendritic forms of poly(amino acids) including polylysine, polyarginine, polyhistidine, and polyglutamate; poly(beta-amino acids) and poly(beta-amino esters); generally cationic amino acids containing peptides and polymers including the class of compounds generally known as ‘cell-penetrating peptides’ (e.g., TAT peptide); aminated polymers derived from water-soluble, uncharged polymers that are modified with particular amine compounds including natural amines such as lysine, histidine, spermine, etc., such as cellulosic materials, polyethyleneglycol and polypropyleneglycol derivatives, polyesters including polyglycolic acid, polylactic acid, polycaprolactone, polyvinyl alcohol, albumin, gelatin, collagen and derivatives thereof, polyacrylates and derivatives thereof, polymethacrylates and derivatives thereof, dextran, cyclodextran, pullulan, chitosan, modified chitosan, carbon based structured materials such as fullerenes and carbon nanotubes, silica, gold, calcium, phosphate and similar inorganic particles; PEI derivatized with silica, polyethyleneglycol, polypropyleneglycol, amino acids, dopamine, poly(2-dimethylaminoethyl methacrylate and derivatives thereof in combination with other polymers to create amphiphilic polymers, spermine, spermidine, pentaethylenehexamine, (N-(2-aminoethyl)-1, 3-propanediamine, N-(3-aminopropyl)-1, 3-propanediamine, tris(2-aminoethyl)amine, N,N′-bis(2aminoethyl)-1, polyamidoamine derivatives with branched or dendritic architectures; and poly(N-(2-hydroxypropyl)methacrylamide) and derivatives thereof. In an exemplary embodiment, the polymer comprises linear, branched, or dendritic forms of PEI.

Suitable lipids include, but are not limited to, aliphatic lipids which may be saturated or unsaturated, and having a carbon chain length ranging from C3 to C22 selected from propanoyl (C3), propanedioyl (C3), pentanedioyl (C5), hexanoic acid or hexanoyl (C6), heptanedioyl (C7), lipoic acid or lipoyl (C8), capryloyl (C8), nonanedioyl (C9), lauric acid or lauroyl (C12), dodecanedioyl (C12), palmitic acid or palmitoyl (C16), stearoyl (C18), linolenoyl (C18), linoleoyl (C18), oleoyl (C18), eicosapentaenoyl (C20), arachidonoyl (C20), eicosanoyl (C20), docosanoyl (22), docosahexaenoyl (22), myristoleic acid (C14:1, cis-9), palmitoleic acid (C16:1, cis-9), stearic acid (C18) and derivatives thereof, oleic acid (C18:1, cis-9), elaidic acid (C18:1, trans-9), linoleic acid (C18:2, cis-9,12) and linolenic acid (C18:3, cis-9,12,15); triglyceride including glyceryl tridecanoate, glyceryl tridodecanoate, glyceryl trimyristate, glyceryl trioctanoate, tripalmitin; lipoic acid and derivatives thereof in oxidized and reduced form; cholesterol and derivatives thereof including cholic acid, deoxycholic acid, and cholanic acid; phospholipids including α-phosphatidylcholine, α-phosphatidylethanolamine, α-phosphatidyl-L-serine, α-phosphatidylinositol, α-phosphatidic acid, α-phosphatidyl-DL-glycerol, α-lysophosphatidylcholine, sphingomyelin, cardiolipin; synthetic lipidic compounds including diphytanoyl phosphatidylethanolamine (DPHPE), dioleoyl phosphatidylethanolamine (DOPE), dioleoyl phosphatidylcholine (DOPC), dilauryl phosphatidylethanolamine (DLPE), 1,2-distearoyl-sn-glycero-3-phosphatidylethanolamine (DSPE), and dimyristoyl phosphatidylethanolamine (DPME); multicyclic lipids and steroids including cholesterol, cholestanol, coprosterol, epicholestanol, epicholesterol, ergostanol, [alpha]-ergostenol, [beta]-ergostenol, [gamma]-ergostenol, ergosterol, 22,23-dihydroergosterol, stigmasterol, stigmastanol, (3[beta])-7-dehydrocholesterol, desmosterol, allocholesterol, 24-hydroxycholesterol, 25-hydroxycholesterol, campesterol, [alpha]-sitosterol, [beta]-sitosterol, [gamma]-sitosterol, lumisterol, pyrocalciferol, isopyrocalciferol, azacosterol, neoergosterol, and dehydroergosterol. In an exemplary embodiment, the lipid comprises an aliphatic lipid.

In one embodiment, the polymeric transfection reagents comprise crosslinked cationic polymers and hydrophobic cationic polymers, each having a combination of cationic groups and lipophilic groups linked via thioester or ester linkages. Such polymers have sufficient cationic charge density to bind nucleic acid by various mechanisms including, but not limited to, electrostatic and hydrophobic interactions, to neutralize the anionic charge of the nucleic acid, and to condense or package the nucleic acid into a form suitable for cell uptake. The interaction of polymers and nucleic acids may result in formation of nanoparticles which are disassembled inside the cells and release the nucleic acid to exert its specific effects upon cell metabolism. Such effects may include, but are not limited to, (i) forced expression of desired genes from DNA or mRNA molecules to produce useful proteins; (ii) forced expression of desired genes to produce non-coding RNAs involved in gene regulation; (iii) silencing of desired mRNAs to stop production of proteins; (iv) silencing of desired regulatory RNAs to interfere with specific gene and mRNA expression; (v) expression of proteins from mRNA or other regulators of intracellular molecules by delivered polynucleotides; and (vi) editing of the genome of a host cell to alter gene expression by delivered polynucleotide complexes. The Examples and Figures herein demonstrate various utilities of the polymeric transfection reagents to achieve such desired outcomes. Such delivery of nucleic acids may be applied in the fields of medicine, biotechnology, and pharmacy.

In the development of the present invention, the inventors prepared PEI transfection reagents by incorporating ester and thioester linkages into low molecular weight PEIs. This is achieved using crosslinkers having variable “carbon-chain length” linkages to yield cationic lipopolymers exhibiting synergistic mechanisms of action including: (i) polycationic groups important for nucleic acid condensation, (ii) hydrophobic groups for increased cell permeability of the delivery system, and (iii) ester and thioester bonding for cleavage at the site of action to promptly release the nucleic acid payload. To obtain higher molecular weight polymers, low molecular weight PEIs may be crosslinked via different covalent bonding schemes including acetal, imine, hydrazine, ester, phosphoester, amide, anhydride, and urethane bonding [49-52]. These materials can undergo stimuli-trigger cleavage which can be exploited to enhance transfection. While the inventors have previously reported the preparation of cationic lipopolymers comprising labile thioester bonds having the formula IA [53], their use in mRNA delivery has not been previously reported. In addition, no thioester crosslinked PEI polymers having the formula IB have been reported to date. As described herein, two types of transfection reagents were developed: i) crosslinked cationic lipopolymers via thioester linkages; and ii) cationic lipopolymers grafted with aliphatic lipids via ester and thioester containing linkers. These cationic lipopolymers undergo degradation via acid-labile linkages (—CO—O— and —CO—S—) and thioester exchange (—CO—S—) reactions.

As described in Examples 1-2, the steps of the process to prepare thioester-containing polymers are as follows. Aliphatic lipid-thioester crosslinkers (i.e., aliphatic lipid crosslinkers comprising thioester linkages) are prepared through substitution reactions whereby one functional group in a chemical compound is replaced by another functional group. In one embodiment, the reaction occurs between a compound comprising a carboxylic acid and a thiol group, and an aliphatic lipid. In one embodiment, the compound comprising a carboxylic acid and thiol group is 3-mercaptopropionic acid. In one embodiment, the aliphatic lipid comprises an aliphatic lipid which may be saturated or unsaturated, and having a carbon chain length ranging from C3 to C22. In one embodiment, the aliphatic lipid comprises an aliphatic acid chloride selected from propanoyl (C3), propanedioyl (C3), pentanedioyl or glutaryl chloride (C5), hexanoic acid or hexanoyl (C6), heptanedioyl or pimeloyl chloride (C7), capryloyl (C8), lipoic acid or lipoyl (C8), nonanedioyl or azelaoyl chloride (C9), lauric acid or lauroyl (C12), dodecanedioyl (C12), palmitic acid or palmitoyl (C16), stearoyl (C18), linoleoyl (C18), oleoyl (C18), eicosanoyl (C20), eicosapentaenoyl (C20), arachidonoyl (C20), linolarachidonoyl (C20), docosanoyl (22), docosahexaenoyl (22), myristoleic acid (C14:1, cis-9), palmitoleic acid (C16:1, cis-9), stearic acid (C18) and derivatives thereof, oleic acid (C18:1, cis-9), elaidic acid (C18:1, trans-9), linoleic acid (C18:2, cis-9,12), orlinolenic acid (C18:3, cis-9,12,15).

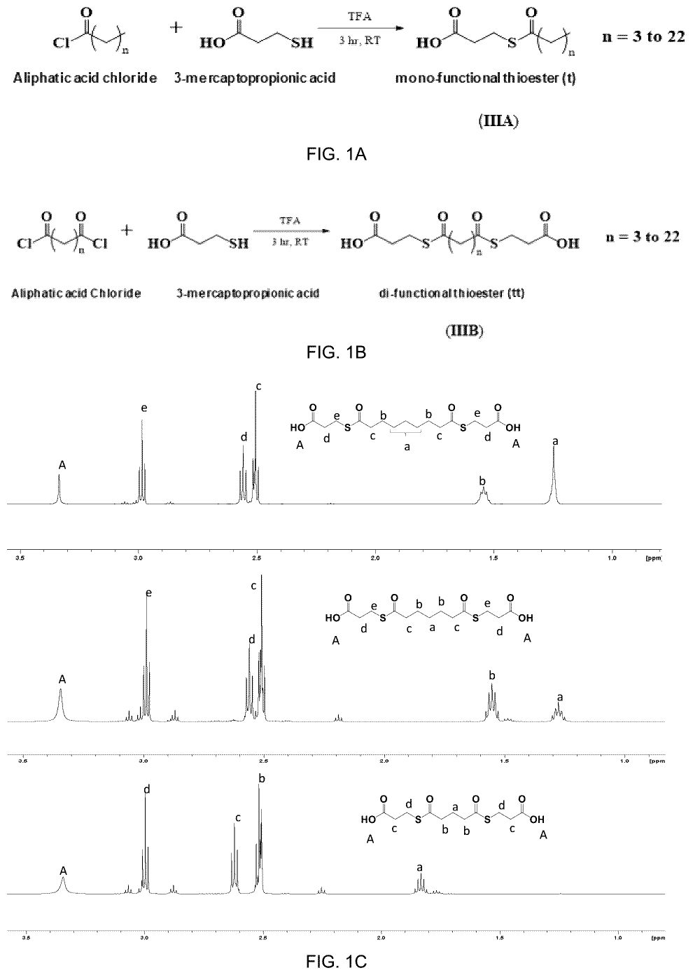

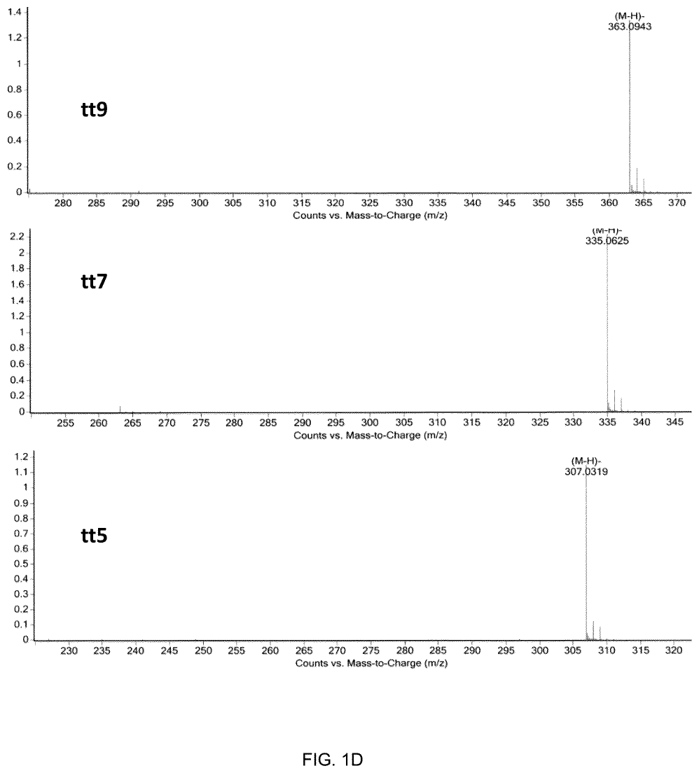

In one embodiment, the aliphatic acid chloride comprises a mono-chloride. In one embodiment shown in FIG. 1A, the aliphatic acid chloride comprising the mono-chloride is reacted with 3-mercaptopropionic acid to yield a mono-functional thioester (designated as “t”). In one embodiment, the mono-functional thioester comprises the compound of formula IIIA:

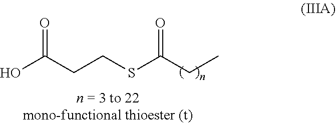

where n is the carbon chain length ranging from C3 to C22.

In one embodiment, the aliphatic acid chloride comprises a di-chloride. In one embodiment shown in FIG. 1B, the aliphatic acid chloride comprising the di-chloride is reacted with 3-mercaptopropionic acid to yield a di-functional thioester (designated as “tt”). In one embodiment, the di-functional thioester comprises the compound of formula IIIB:

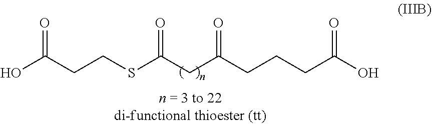

where n is the carbon chain length ranging from C3 to C22.

In the reactions shown in FIGS. 1A-B, the aliphatic acid chloride and 3-mercaptopropionic acid are dissolved separately in trifluoroacetic acid (Example 2). The 3-mercaptopropionic acid solution is added to the aliphatic acid chloride solution and the mixture is stirred for three hours at room temperature. The final product is precipitated in hexane/diethyl ether and dried under vacuum. In one embodiment, the final product comprises an aliphatic lipid end-capped with mono- or di-carboxyl functionality via thioester bonding.

As used herein, the term “polyethylenimine” (“PEI”) means a polymer with a repeating unit composed of the amine group and two carbon aliphatic CH₂CH₂ spacers. The term is meant to include linear, branched, or dendritic forms. The term is meant to include linear polyethylenimines (“lPEI”) containing all secondary amines and terminal primary amines; branched polyethylenimines (“bPEI”) which contain primary, secondary and tertiary amino groups; and hyperbranched, dendritic forms with primary, secondary and tertiary amino groups.

In one embodiment, PEI is selected from a lPEI or a bPEI (where each of x and y in PEI reaction schemes ranges between 5 and 30) or PEI which may be derived from, for example, ethyleneimine or other similar building block as shown below:

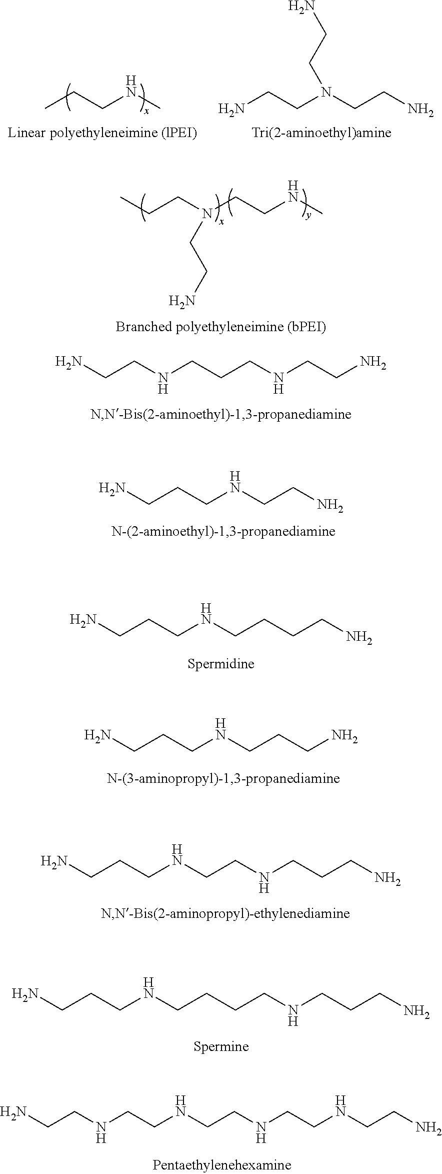

In one embodiment, PEI has a low molecular weight ranging from about 0.5 kDa to about 5 kDa, and more preferably from about 0.6 kDa to about 2.5 kDa. Without being bound by any theory, the low molecular weight may reduce the strength of the binding between a polymer and nucleic acid, thus ensuring that nucleic acid can be easily and readily released once inside a cell. The thioester of the crosslinker and lipid groups may also reduce the binding strength.

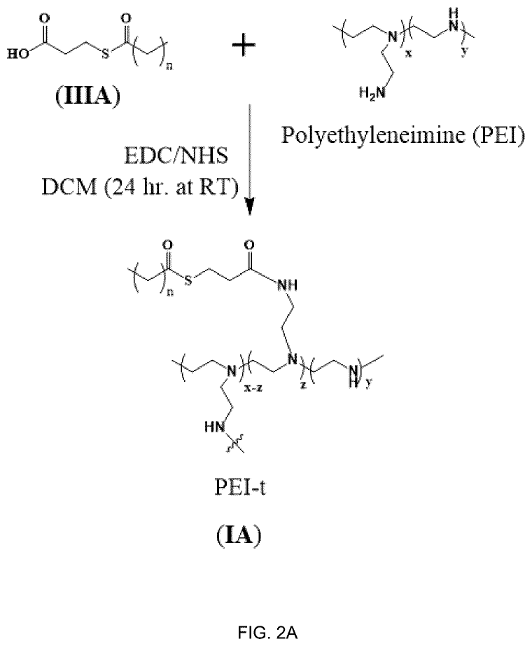

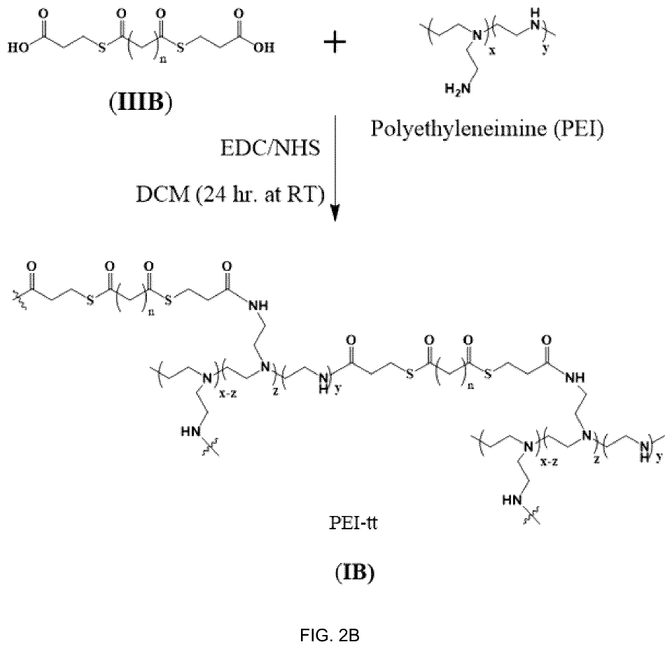

The aliphatic lipid-thioester crosslinkers and PEIs are used as starting materials for preparing transfection reagents (Example 2, Table 1). In one embodiment of a reaction shown in FIG. 2A, the mono-functional thioester (t) of formula IIIA and PEI are used to prepare a cationic lipopolymer. In one embodiment, the mono-functional thioester (t) of formula IIIA is grafted onto the PEI. In one embodiment, the transfection reagent (designated as “PEI-t” to refer to a cleavable hydrophobic derivative of PEI) comprises a compound of formula IA:

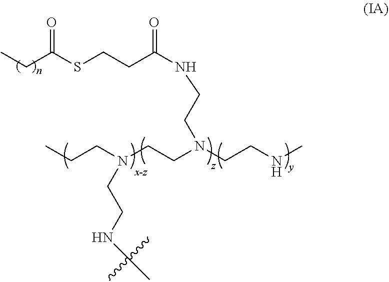

wherein the hydrophobic group comprises 3<n<22 atoms; x=5<n<30; y=5<n<30; and z=1<n<5.

In one embodiment of a reaction shown in FIG. 2B, the di-functional thioester (tt) of formula IIIB and PEI are used to prepare a cationic lipopolymer. In one embodiment, the di-functional thioester (tt) of formula IIIB is crosslinked onto PEI. In one embodiment, the transfection reagent (designated as “PEI-tt” to refer to a cleavable crosslinked PEI) comprises a compound of formula IB:

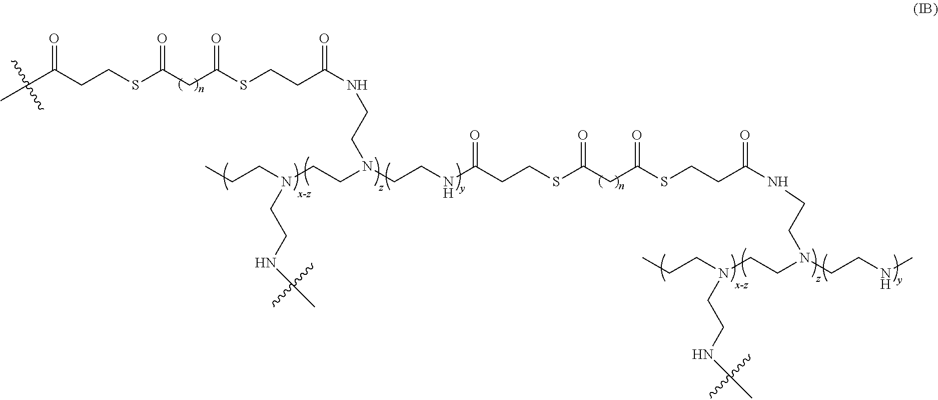

wherein the linker comprises 3<n<22 atoms; x=5<n<30; y=5<n<30; and z=1<n<5.

In the reactions shown in FIGS. 2A-B, the aliphatic lipid-thioester crosslinkers are either grafted or crosslinked onto branched PEIs through 1-ethyl-3-(3-dimethylamino-propyl)carbodiimide (EDC)/N-hydroxysuccinimide (NHS) activation (Example 2). The structural composition of the compounds of formula IA and IB may be confirmed by examination of one or more spectra including, but not limited to, ¹H-NMR spectroscopy, infrared spectra, and mass spectra (Example 2). Crosslinking can alter the buffering capacity of PEIs due to the change in composition. The buffering capacity of PEI-tt polymers may be determined by acid-base titration (Example 4). The transfection reagent may be examined for DNA binding, unpacking and digestion using a dye exclusion and an agarose gel retardation assay (Example 5).

As described in Example 3, transfection reagents may be prepared comprising cationic lipopolymers grafted with aliphatic lipids via ester-containing linkers or thioester-containing linkers through EDC/NHS activation. In one embodiment, the aliphatic lipid-ester or lip-thioester crosslinker has the formula IVA, IVB, IVC, or IVD.

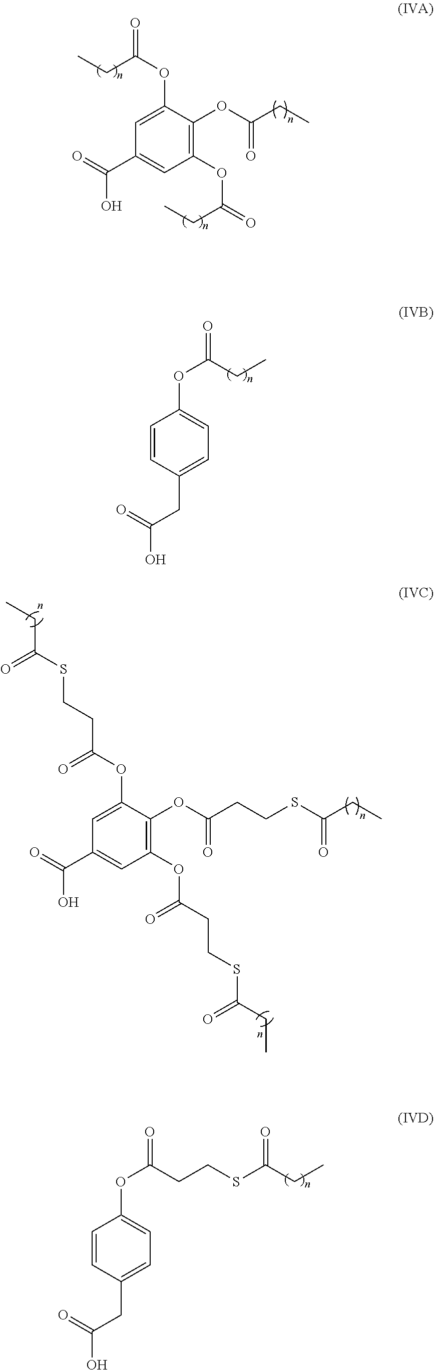

where n is the carbon chain length ranging from C3 to C22.

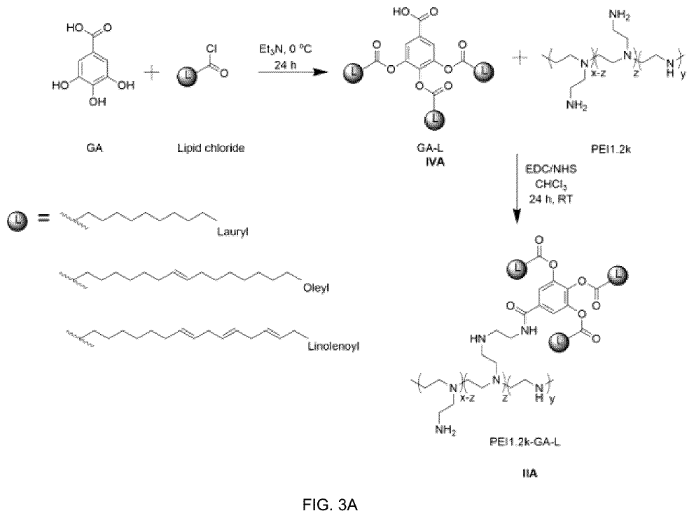

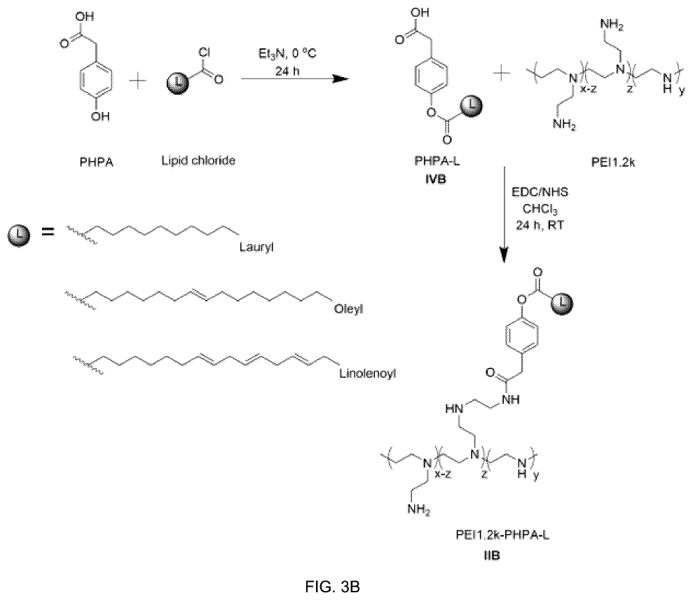

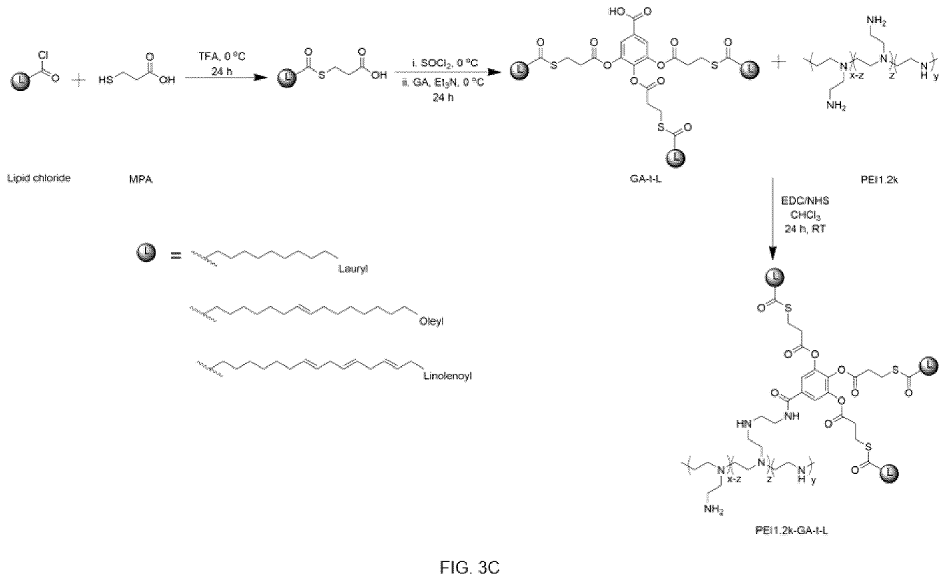

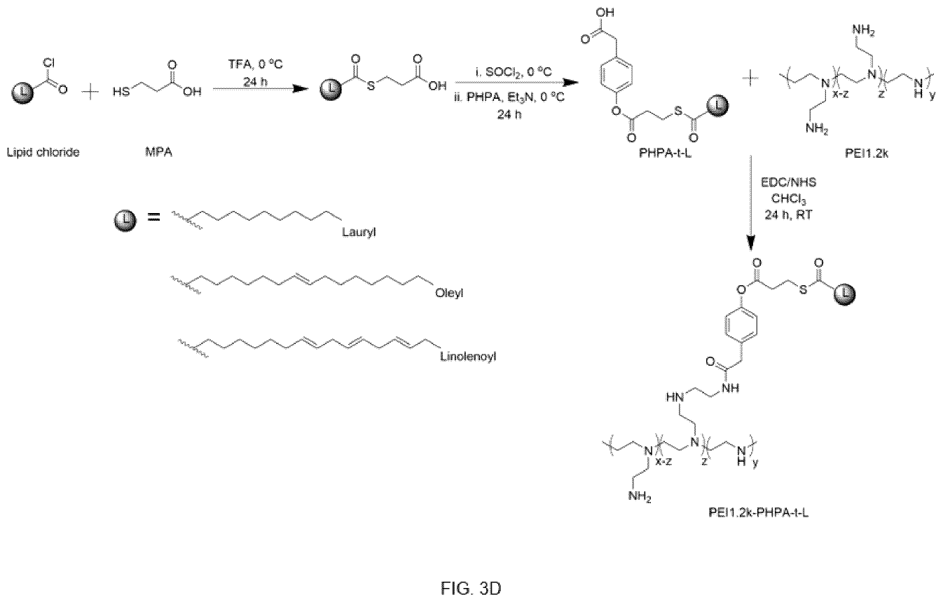

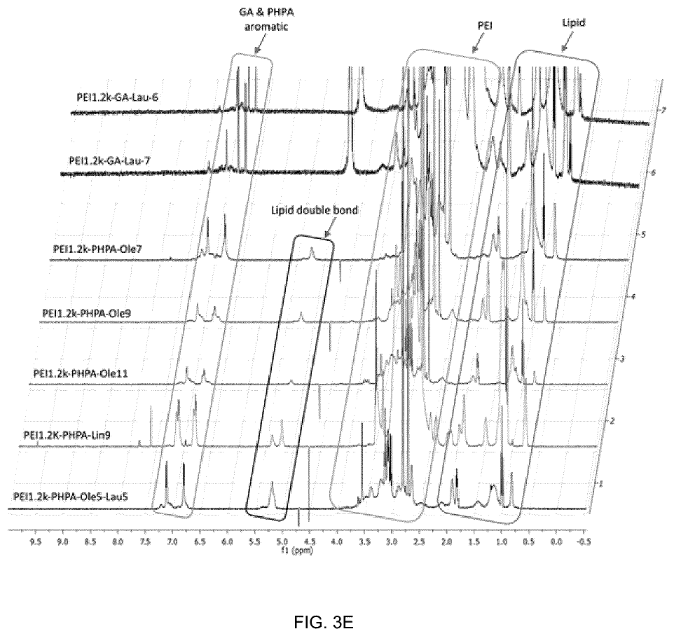

In one embodiment of a reaction shown in FIG. 3B, a mono-functional linker is used to prepare a cationic lipopolymer via an ester or thioester linkage. In one embodiment, 4-hydroxyphenylacetic acid (PUPA) is reacted with a lipid chloride to yield mono-functional PHPA-L of formula IVB. PHPA-L is grafted onto PEI with an ester linkage to yield a compound having formula IIB:

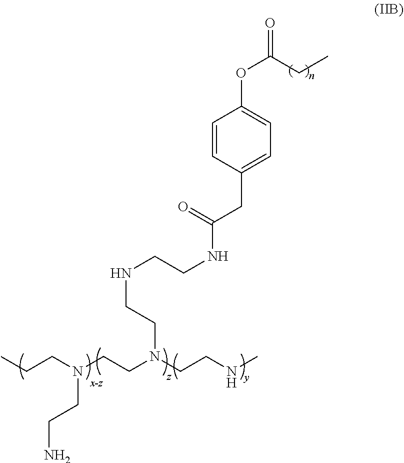

where the compound comprises a carbon chain length of 3<n<22 atoms; x=5<n<30; y=5<n<30; and z=1<n<5.

In one embodiment of a reaction shown in FIG. 3D, the mono-functional linker PUPA is reacted with a lipid chloride to yield mono-functional PHPA-L of formula IVD with a thioester linkage. PHPA-L is grafted onto PEI to yield a compound having formula IID that bears a thioester linkage:

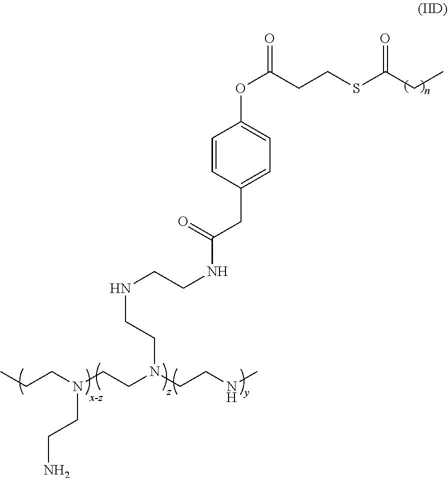

where the compound comprises a carbon chain length of 3<n<22 atoms; x=5<n<30; y=5<n<30; and z=1<n<5.

In one embodiment of a reaction shown in FIG. 3A, a tri-functional linker is used to prepare a cationic lipopolymer via an ester or thioester linkage. In one embodiment, gallic acid (GA) is reacted with a lipid chloride to yield GA-L of formula IVA with an ester group. GA-L is grafted onto PEI to yield a compound having formula IIA:

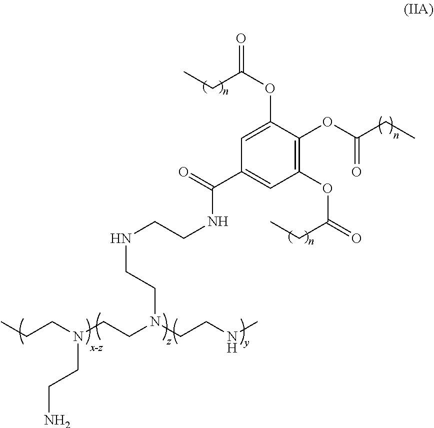

where the compound comprises a carbon chain length of 3<n<22 atoms; x=5<n<30; y=5<n<30; and z=1<n<5.

In one embodiment of a reaction shown in FIG. 3C, the tri-functional linker gallic acid (GA) is reacted with a lipid to yield a GA-L compound of formula IVC with a thioester group. The GA-L is then grafted onto PEI to yield a compound having formula IIC:

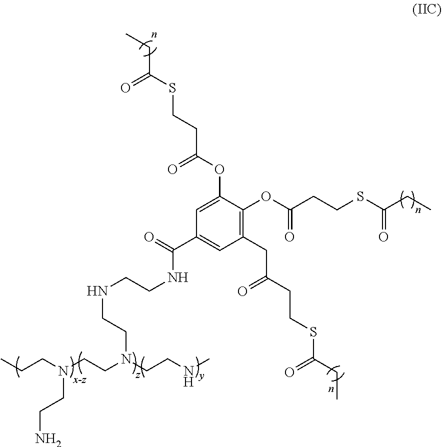

where the compound comprises a carbon chain length of 3<n<22 atoms; x=5<n<30; y=5<n<30; and z=1<n<5.

In one embodiment, the invention comprises a nanoparticle comprising the compound of formula IA, IB, IIA, IIB, IIC, or IID complexed to a biologically active nucleic acid and either with or without an additive to prepare the following complexes (VA) and (VB):

As used herein, the term “nucleic acid” means a polynucleotide such as deoxyribonucleic acid (DNA), and, where appropriate, ribonucleic acid (RNA). The term should also be understood to include, as equivalents, analogs of either RNA or DNA. As used herein, the term “polynucleotide” is a linear sequence of ribonucleotides (RNA) or deoxyribonucleotides (DNA) in which the 3′ carbon of the pentose sugar of one nucleotide is linked to the 5′ carbon of the pentose sugar of another nucleotide. The deoxyribonucleotide bases are abbreviated as “A” deoxyadenine; “C” deoxycytidine; “G” deoxyguanine; “T” deoxythymidine; “I” deoxyinosine. Suitable nucleic acids for delivery by the transfection reagents of the present invention include, but are not limited to, DNA-based nucleic acids (e.g., a DNA-based oligonucleotide or antisense oligonucleotide, plasmid DNA for encoding an RNA product comprising short hairpin RNA (shRNA), mRNA for protein synthesis, sgRNA, or combinations thereof); RNA-based nucleic acids (e.g., short interfering RNA (siRNA) such as, for example, synthetic siRNA intended to silence endogenous gene expression; single guide RNA (sgRNA) such as, for example, sgRNA for genome editing; microRNA; mRNA such as, for example, mRNA for encoding protein; short hairpin RNA (shRNA); or combinations thereof); a peptide-nucleic acid (PNA); DNA-RNA chimeras; and nucleic acids in combination with proteins for example, for use in genome editing. In one embodiment, the nucleic acid comprises one or more of plasmid DNA with genes of interest and transposases. As used herein, the term “additive” means a compound including, but not limited to, a neutral or anionic additive selected from polyanions, polyacrylic acid, polymethacrylic acid, polyaspartic acid, polyglutamic acid, gelatin, hyaluronic acid, cellulose, or derivatives thereof.

The nanoparticle can be analyzed to determine its physical and chemical properties. In one embodiment, the nanoparticle has a hydrodynamic size ranging from about 50 nm to about 200 nm, and preferably from about 100 nm to about 200 nm. Such hydrodynamic sizes are considered sufficiently small so as to be suitable for effective cellular uptake. In one embodiment, the nanoparticle has a surface charge or C-potential which has been enhanced in the range of about +0 mV to about +35 mV, and more preferably about −10 mV to +0 mV.

In one embodiment, the nanoparticle comprises a compound (for example, of formula IA, IB, IIA, IIB, IIC, or IID) and nucleic acid to yield a polymer/nucleic acid binary complex of formula VA. In one embodiment, a nucleic acid solution is added to the compound in water or an aqueous-based buffer, and incubated for 30 minutes at room temperature to yield the polymer/nucleic acid binary complex (Example 6). The use of water or an aqueous-based buffer generates the polymer/nucleic acid binary complex in the form of a nanoparticle, eliminating the need to use cell-toxic organic solvents during nanoparticle formation.

In one embodiment, the nanoparticle comprises a compound (for example, of formula IA, IB, IIA, IIB, IIC, or IID), nucleic acid, and additive to yield a polymer/nucleic acid ternary complex of formula VB. In one embodiment, nucleic acid and an additive are mixed together and added to the compound of formula IA, IB, IIA, IIB, IIC, or IID in water or an aqueous-based buffer, and incubated for 30 minutes at room temperature to yield the polymer/nucleic acid ternary complex (Example 7). The use of water or an aqueous-based buffer generates the polymer/nucleic acid ternary complex in the form of a nanoparticle, eliminating the need to use cell-toxic organic solvents during nanoparticle formation.

The functionalities of the polymer/nucleic acid binary and ternary complexes may be confirmed by testing in various ways including in vitro cell culture assays using appropriate host cells, meaning any cell type that can be transfected with present invention (Examples 6 and 8). The polymer/nucleic acid binary and ternary complexes can be introduced into host cells by various techniques for transfection. As used herein, the term “transfection” refers to the uptake of exogenous nucleic acid (for example, DNA or RNA) by a cell by any means practicable. The uptake of nucleic acid results in a transient transfection regardless of the means by which the uptake is accomplished. Those skilled in the art can select a particular host cell line that is best suited to assess expression of a gene of interest.

Suitable host cells include, but are not limited to, anchorage-dependent cells, anchorage-independent cells, and easy-to-grow cell lines typically used for production of various biochemicals including proteins. The term “anchorage-dependent cell” means a cell which needs contact and anchorage to a stable surface to grow, function, and divide. The term “anchorage-independent cell” means a cell which has lost the need for anchorage dependence and has transformed to grow without attaching to a substrate, and thus is typically difficult to transfect. Examples of anchorage-independent cells include, but are not limited to, hematopoietic cells. As used herein, the term “hematopoietic cells” refers to cells which can develop into all different types of functional blood cells in lines known as myeloid and lymphoid. As used herein, the term “myeloid cells” includes megakaryocytes, thrombocytes, erythrocytes, mast cells, myeloblasts, basophils, neutrophils, eosinophils, monocytes, and macrophages. As used herein, the term “lymphoid cells” includes small lymphocytes (i.e., T-cells including helper T-cells, and B-cells) and large granular lymphocytes (Natural Killer cells) typically found in circulating blood, bone marrow and other parts of the lymphatic system such as the spleen and lymph nodes. The ability of the polymeric transfection reagents of the present invention to function as effective DNA or RNA transfection reagents to target anchorage-independent cells is a considerable advantage in various medical conditions. In an exemplary embodiment, lymphoid cells were transfected with exogenous gene expression systems, transforming the lymphoid cells into cancer cell reactive phenotype.

All materials used in the present invention are non-toxic, inexpensive, readily available, and compatible with highly sensitive cells. Here, the use of compatible materials which are non-toxic and otherwise non-damaging to humans or human tissues, is intended to render the compounds and compositions of the present invention suitable for human utility.

In one embodiment, the invention comprises a composition or pharmaceutical composition comprising the nanoparticle and a pharmaceutically acceptable carrier. As used herein, the term “carrier” means a suitable vehicle which is biocompatible and pharmaceutically acceptable, including, for instance, liquid diluents which are suitable for administration. Those skilled in the art are familiar with any pharmaceutically acceptable carrier that would be useful in this regard, and therefore the procedure for making pharmaceutical compositions in accordance with the invention will not be discussed in detail.

In one embodiment, the invention comprises a method of treating, preventing, or ameliorating a disease in a subject, comprising administering to the subject an effective amount of the nanoparticle, composition or pharmaceutical composition. As used herein, the term “disease” includes, but is not limited to, any disease including, but not limited to, chronic and acute myeloid leukemia, chronic and acute lymphocytic leukemia, hairy cell leukemia, meningeal leukemia, myeloma, multiple myeloma, lymphoma, brain cancer, bladder cancer, breast cancer, melanoma, skin cancer, epidermal carcinoma, colon and rectal cancer, lung cancer, non-Hodgkin lymphoma, Hodgkin lymphoma, Sezary Syndrome, endometrial cancer, pancreatic cancer, kidney cancer, prostate cancer, leukemia thyroid cancer, head and neck cancer, ovarian cancer, hepatocellular cancer, cervical cancer, sarcoma, gastric cancer, gastrointestinal cancer, uterine cancer, and the like.

In one embodiment, the invention comprises a method of treating, preventing, or ameliorating a disease in a subject through genetically modified hematopoietic host cells, particularly lymphoid cells, comprising administering to a subject an effective amount of the nanoparticle, composition or pharmaceutical composition. In one embodiment, the host lymphoid cells are selected from T-cells or Natural Killer cells. In one embodiment, the T-cells are helper T-cells. In one embodiment, the nanoparticle, composition or pharmaceutical composition comprises a nucleic acid selected from DNA or RNA. As used herein, the term “subject” means a human or other vertebrate. As used herein, the term “effective amount” means any amount of a formulation of the nanoparticle useful for treating, preventing, or ameliorating a disease or disorder upon administration. An effective amount of the composition provides either subjective relief of symptoms or an objectively identifiable improvement as noted by the clinician or other qualified observer. As used herein, the terms “treating,” “preventing” and “ameliorating” refer to interventions performed with the intention of alleviating the symptoms associated with, preventing the development of, or altering the pathology of a disease, disorder or condition. Thus, in various embodiments, the terms may include the prevention (prophylaxis), moderation, reduction, or curing of a disease, disorder or condition at various stages. In various embodiments, therefore, those in need of therapy/treatment may include those already having the disease, disorder or condition and/or those prone to, or at risk of developing, the disease, disorder or condition and/or those in whom the disease, disorder or condition is to be prevented.

In one embodiment, an effective amount of the nanoparticle or a composition comprising same can be administered to the subject in conjunction with one or more drugs used to treat the disease to provide complementary activity. Careful selection of conventional drug therapy combined with the nanoparticle and compositions of the present invention may enhance the therapeutic response to either treatment approach.

In one embodiment, the invention comprises use of the nanoparticle, composition or pharmaceutical composition to treat, prevent, or ameliorate a disease in a subject. In one embodiment, the invention comprises use of the nanoparticle, composition or pharmaceutical composition to treat, prevent, or ameliorate a disease in a subject through genetically modified hematopoietic host cells, particularly lymphoid cells. In one embodiment, the host lymphoid cells are selected from T-cells or Natural Killer cells. In one embodiment, the T-cells are helper T-cells. In one embodiment, the nanoparticle, composition or pharmaceutical composition comprises a nucleic acid selected from DNA or RNA.

In one embodiment, the invention comprises a method of delivering mRNA or a ribonucleotide protein complex (RNP) using a compound having the formula IA, IB, IIA, IIB, IIC, or IID. In one embodiment, the compound has the formula IA. As described in Example 11, RNP complexes may be delivered to hosts cells for gene editing.

Embodiments of the present invention are described in the following Examples, which are set forth to aid in the understanding of the invention and should not be construed to limit in any way the scope of the invention as defined in the claims which follow thereafter.

### Example 1—Materials

Branched polyethylenimine (PEI) of 1.2 kDa (PEI1.2), 0.6 kDa (PEI0.6) and linear polyethylenimine (lPEI) of 2.5 kDa (lPEI2.5) and 40 kDa (lPEI40) were obtained from Polysciences, Inc. (Warrington, PA, USA) and used without any purification. Mercaptopropionic acid (MPA), aliphatic lipids (glutaryl chloride; C5, pimeloyl chloride; C7, and azelaoyl chloride; C9), 3-(4,5-Dimethylthiazol-2-yl)-2,5-diphenyltetrazolium bromide (MTT), branched polyethylenimine (PEI) of 2 kDa (PEI2) [50%, w/v in water], and trypsin/EDTA were obtained from Sigma-Aldrich Corporation (St Louis, MO). SYBR Green I was purchased from Cambrex Bio Science (Rockland, MD). Cell culture medium, RPMI 1640, supplied with L-glutamine and 25 mM HEPES, and Penicillin (10.000 U/mL)/Streptomycin (10 mg/mL) were obtained from Invitrogen (Grand Island, NY). Fetal bovine serum (FBS) was purchased from PAA Laboratories Inc. (Etobicoke, ON). The plasmids gWIZ (blank plasmid with CMV promoter) and gWIZ-GFP (AcGFP expressing plasmid with CMV promoter) used in transfection studies were purchased from Aldevron (Fargo, ND). All RNA molecules can be obtained from commercial vendors by custom synthesis. The solvents were obtained from Sigma-Aldrich and used without any further purification.

### Example 2—Synthesis and Characterization of Thioester Polymers

Thioester crosslinked PEIs were synthesized via N-acylation of aliphatic lipids (C5, C7 and C9) (II). Briefly, aliphatic lipid (e.g., glutaryl chloride) (169.01 μL, 1.0 mmol) and MPA (332 μL, 2.5 mmol) were separately dissolved in trifluoroacetic acid (600 μL), MPA solution was slowly added to the lipids solution, and the reaction was stirred for 3 hr. at room temperature. The carboxyl end-capped aliphatic lipids (tt5, tt7, tt9) were collected by precipitation (3×) in ice cold hexane/diethyl ether and dried under vacuum for 48 hr. at room temperature. The tts were then employed to crosslink PEIs (PEI-tt) through EDC/NHS activation. Briefly, tt (0.1 mmol in 20 mL CHCl₃) was activated with EDC (0.15 mmol in 1 mL CHCl₃) and NHS (0.15 mmol in 1 mL methanol) at room temperature for 1 hr. The activated TTs were added dropwise to PEIs solution (0.1 mmol in 100 mL CHCl₃) and the reaction mixture was stirred overnight at room temperature. The crude product was recovered by precipitation (3×) in ice cold diethyl ether and dried under vacuum for 48 hr. As a control, PEI1.2 crosslinked with acid chlorides (C5, C7 and C9) via amide bonding was used [55]. Structural compositions of tts and PEI-tt were elucidated through ¹H-NMR spectroscopy (Bruker 300 MHz, Billerica, MA) using CDCl₃ and D₂O as solvents, and molecular weight by mass spectroscopy and MALDI-TOF.

TABLE 1
Summary of polymers synthesized with thioester crosslinkers

| | | | Feed Ratio, Cross- |
|---|---|---|---|
| SN | Polymer | Cross-Linker | Linker/Polymer (mol/mol) |
| 1 | PEI-tt5-1 | tt5 | 1 |
| 2 | PEI-tt5-2 | tt5 | 2 |
| 3 | PEI-tt7-1 | tt7 | 1 |
| 4 | PEI-tt7-2 | tt7 | 2 |
| 5 | PEI-tt9-1 | tt9 | 1 |
| 6 | PEI-tt9-2 | tt9 | 2 |

List of Polymers

| | | | |
|---|---|---|---|
| PEI0.6-tt5-1 | PEI1.2-tt5-1 | PEI2-tt5-1 | IPEI-tt5-1 |
| PEI0.6-tt5-2 | PEI1.2-tt5-2 | PEI2-tt5-2 | IPEI-tt5-2 |
| PEI0.6-tt7-1 | PEI1.2-tt7-1 | PEI2-tt7-1 | IPEI-tt7-1 |
| PEI0.6-tt7-2 | PEI1.2-tt7-2 | PEI2-tt7-2 | IPEI-tt7-2 |
| PEI0.6-tt9-1 | PEI1.2-tt9-1 | PEI2-tt9-1 | IPEI-tt9-1 |
| PEI0.6-tt9-2 | PEI1.2-tt9-2 | PEI2-tt9-2 | IPEI-tt9-2 |

### Example 3—Synthesis and Characterization of Ester Polymers

Preparation of ester-linked lipopolymers via gallic acid and p-hydroxyphenylacetic acid (PEI1.2k-GA-L and PEI1.2k-PHPA-L, respectively) may be conducted in two steps. In the first step of preparing GA-L or PHPA-L, the corresponding lipid chloride was added dropwise into the cooled (0° C.) solution of GA or PHPA and Et₃N in acetone (5 mL) and stirred overnight on ice. Acetone was then evaporated, and the mixture was diluted with CH₂CL₂ (10 mL). The Et₃N·HCl salt was filtered, and the filtrate was washed with saturated aqueous NaHCO₃, water, and then dried (MgSO₄). The organic solvent was removed by rotary evaporation to yield the final products GA-L or PHPA-L. In the second step of preparing the final lipopolymers, EDC in CHCl₃ (1 mL) was added with GA-L or PHPA-L in CHCl₃ (3 mL) and stirred for 1 hr at room temperature. Subsequently, NHS in 0.5 mL MeOH was added into these solutions and the stirring was continued for another 1 hr. The activated GA-L solution was then added into PEI solutions in CHCl₃ (50 mg PEI in 50 mL CHCl₃) and the reaction was stirred continuously for 24 hrs. The solvent was evaporated, and the concentrated solution was precipitated in ice cold diethyl ether (3×). The precipitate was centrifuged and freeze-dried for 48 hrs to obtain white powder as the product. The GA-L and PHPA-L intermediates and the resultant lipopolymers were analyzed for composition using ¹H-NMR (Bruker 300 MHz, Billerica, MA).

### Example 4—Acid-Base Titration

Buffering capacity of the polymers may be determined by acid-base titration [55]. Briefly, a polymer solution (0.2 mg/mL) is prepared in 0.15 M NaCl and the pH set to 10 using aqueous NaOH (0.1 M). The solution is titrated from pH 10 to 2 with HCl (0.1 M). As a control experiment, the solution of parent polymers (0.2 mg/mL, in 0.15 M NaCl) is titrated. The change of pH with parent polymers is more gradual as compared to titrating the solution without any polymers. With modified polymers, the buffering capacity may be reduced to some extent (e.g., 10%), but remains similar to the change of pH of parent polymers with HCl addition.

### Example 5—Dye Exclusion Assay

DNA binding capacity of the polymers was measured through a dye exclusion assay [55]. Briefly, DNA (4 μL, 25 μg/mL) was added to a polymer solution diluted in ddH₂O in the concentration range of 0.1 to 4 μg/to generate complexes of mass ratios 0.025 to 1.0. After 30 minutes of incubation at room temperature, 300 μL of SYBR green I (1×) was added to each tube and 100 μL of each sample was read on a 96-well plate (Fluoroskan Ascent; Thermo Labsystems) at λ_{EX} of 485 nm and λ_{EM} of 527 nm to quantify the amount of free DNA left. The fluorescence values obtained in triplicate were normalized with the fluorescence of free DNA solution (i.e., in the absence of polymers) and plotted as a function of polymer/pDNA ratio.

### Example 6—Cell Culture

Attachment-independent lymphoid cells (Jurkat) were used to model human T-cells. Cells were maintained in RPMI (C-L cells) medium containing FBS (10%), penicillin (100 U/mL) and 100 μg/ml streptomycin in a humidified atmosphere of 95 air/5% CO₂. They were routinely cultured on T75 cell culture flask. Reverse transfection was performed in the cells seeded (100,000 cells/mL) in 48-well plates. Other cell types used to assess the functionality of various nucleic acids included breast cancer MDA-MB-436 cells, human kidney epithelial 293T cells, and African green monkey kidney epithelial Vero cells.

### Example 7—Toxicity Study

Cellular toxicity of polymer/DNA complexes was assessed in Jurkat cells. Polymer/DNA complexes ratio 5.0, w/w (group PEI1.2, PEI2.0 and lPEI2.5) and ratio 15.0 (group PEI0.6) were prepared in serum free RPMI medium at room temperature and directly added to the cells. Briefly, 3.0 μL (0.4 μg/μL) of giWIZ-GFP was mixed with 6.0 μL (1 mg/mL) of polymer in 300 μL RPMI to yield complexes of ratio 5.0, w/w. After 30 min incubation at room temperature, complexes (100 μL) were directly transferred to a 48-well plate. 300 μL (100,000 cells/mL) of cells was added on top of the wells and incubated in the humidified atmosphere of 95% air/5% CO₂. The cell growth was assessed on day-2 via the MTT (3-(4,5-dimethylthiazol-2-yl)-2,5-diphenyl-tetrazolium bromide) assay. MTT reagent (5 μg/μL in HBSS) was directly added to the wells to yield a final concentration of 1.0 μg/μL and incubated for 3 hr. in the humidified atmosphere of 95% air/5% CO₂. The cells were collected in microcentrifuge tubes (1.5 mL) and centrifuged at 1400 rpm for 5 min., washed (2×) with HBSS (pH 7.4.) and formazan crystal was dissolved in DMSO (200 μL). The optical density was measured in a universal microplate reader (ELx, Bio-Tech Instrument, Inc.) at X=570 nm. The cells without any treatment were used as reference and cell viability was expressed as a percentage of this reference control.

### Example 8—Transfection Study

The Jurkat cells were treated with polymer/DNA complexes or polymer/mRNA (prepared similarly to the protocol above) and incubated for 72 hr. in a humidified atmosphere at 37° C. The cells were processed for flow cytometry to quantify the extent of GFP expression, cells were processed for flow cytometry and GFP levels in the cells were quantified using FL1 channel in the Beckman Coulter QUANTA™ SC Flow Cytometer. The results are expressed as the percentage of reduction in GFP fluorescence levels and percentage of cells that displayed reduced GFP levels as compared to untreated cells.

### Example 9—Cell Killing Study

Blood was obtained from two healthy donors and peripheral blood mononuclear cells (PBMCs) were isolated by centrifugation at 210 g and cultured in RPMI medium containing 10% serum, antibiotics and IL-2 (100 U/mL) to enrich for T-cell lymphocytes. The cells were treated with polymer/DNA complexes or polymer/mRNA (prepared similarly to the protocol above) and incubated for 24 hr in a humidified atmosphere at 37° C. The pDNA and mRNA were designed to express chimeric antigen receptors (CARs) against human and mouse CD19. The cells were then mixed with human CD19(+) RS4;11 cells and mouse CD19(+) WEHI cells at effector:target (E:T) ratio of 5:1 and cultured for another 5 days. The CD19(+) cell population was then determined by staining for the mouse and human CD19 with specific antibodies and measuring the percentage of CD19(+) cells by flow cytometry.

### Example 10—Testing in an Animal Model

All experiments were conducted in accordance with pre-approved protocols by the Health Sciences Laboratory Animal Services (University of Alberta). To create breast cancer xenografts, 12-14 weeks old female NOD.Cg.Prkdc(Scid)II2rg mice were anesthetized using isoflurane and ˜3 million SUM-149 cells in Matrigel™ and DMEM (1:1) were injected subcutaneously. Tumor growth was monitored every 96 h and tumor length and width were measured using a digital caliper to calculate the volume by the formula <length×width²×0.5>. The mice were divided into 3 groups: no treatment, mock treatment with a GFP coding mRNA (mGFP) and a TRAIL coding mRNA (mTRAIL). The mRNAs were mixed with the Polymer IA in DMEM using a mass ratio of 1:5, respectively. Once the tumor was developed, 40 μL of polymer/mRNA complexes (w/w ratio 5:1) were injected subcutaneously to tumor vicinity. Four injections were performed with 96 h apart (5-5-3-3 μg mRNA per injection). After 48 h of last injection, mice were euthanized, tumors were collected and weighted.

### Example 11—Gene Editing Study

GFP-expressing MDA-MB-231 cells were seeded in a 96-well plate at a density of 10,000 cells/well and allowed to attach overnight. Cas9/sgRNA complexes were prepared using Alt-R® spCas9 Nuclease V3 (IDT) and CRISPRevolution synthetic sgRNA (Synthego) in RPMI medium at a molar ratio of 3:1 sgRNA:Cas9. Two sets of complexes were prepared using non-specific sgRNA and sgRNA targeting the GFP coding sequence, Complexation for RNP formation was allowed for 30 minutes, after which lipid-substituted polymers were incubated with RNP (5:1 w/w ratio) for 30 minutes at room temperature. RNP complexes were added to the cells and incubated under standard cell culture conditions for 5 days. Flow cytometry was used to analyze cells for knock-out of GFP expression.

### Example 12—Results of Examples 1-11

### Crosslinker Synthesis and Characterization

In a ¹H-NMR spectra of tt-crosslinkers, characteristic chemical shifts corresponding to protons (—CH₂—) of aliphatic chain a (δ ˜1.35 ppm), b (δ ˜1.5 ppm) and c (δ ˜2.5 ppm) were observed along with the protons (—CH₂—) of 3-mercaptopropionic acid, e (δ ˜3.5 ppm), and d (δ ˜2.6 ppm) (FIG. 1C). The resonance peaks of ethyl protons of both lipids “c” and 3-mercaptopropionic acids “e” were significantly shifted which indicate thiol substitution onto carbonyl carbon of lipid molecules; therefore, the integrated values of these protons (c, δ ˜2.5 ppm and e, δ ˜3.5 ppm) were used to quantify the structural composition of the product which was 1:2, lipids: mertcapto-ethyl. The end capping was confirmed by mass spectroscopy (FIG. 1D). Each molecule was composed of one molar mass (tt5: 307, tt7: 335, tt9: 363) of lipids and two molar mass of 3-mercaptopropionic acids, indicating that two molecules of mercapto-ethyl were substituted at the distal ends of the lipid molecules.

### Polymer Synthesis and Characterization

Aliphatic lipids end-capped with carboxyl group via thioester functionality are incorporated onto PEIs via EDC/NHS activation (FIGS. 2A-B). For the incorporation of di-functional aliphatic lipids (tt), the content of tt-crosslinkers in feed ratio 1:1 and 1:2 (PEI:TT, mol:mol) was employed using a slightly modified protocol. The EDC/NHS activation is similarly used to incorporate lipids to PEI polymers with ester linkages of PUPA and GA (FIGS. 3A-D). Successful lipid incorporation is confirmed with ¹H-NMR (FIG. 3E) where the expected linker identities on the generated spectra (as indicated on FIGS. 3A-B) are confirmed.

### Buffering Capacity

Without being limited to any theory, the transfection efficiency of PEIs based polymers may be driven by an efficient endosomal escape which is facilitated by their extraordinary buffering capacity under endosomal pH condition. All the tested polymers (e.g., PEI-tt) showed nearly same buffering capacity on titration from pH 10 to 2 and it was identical to the parent polymers, indicating the negligible effect of tt-incorporation.

### DNA Binding Efficiency

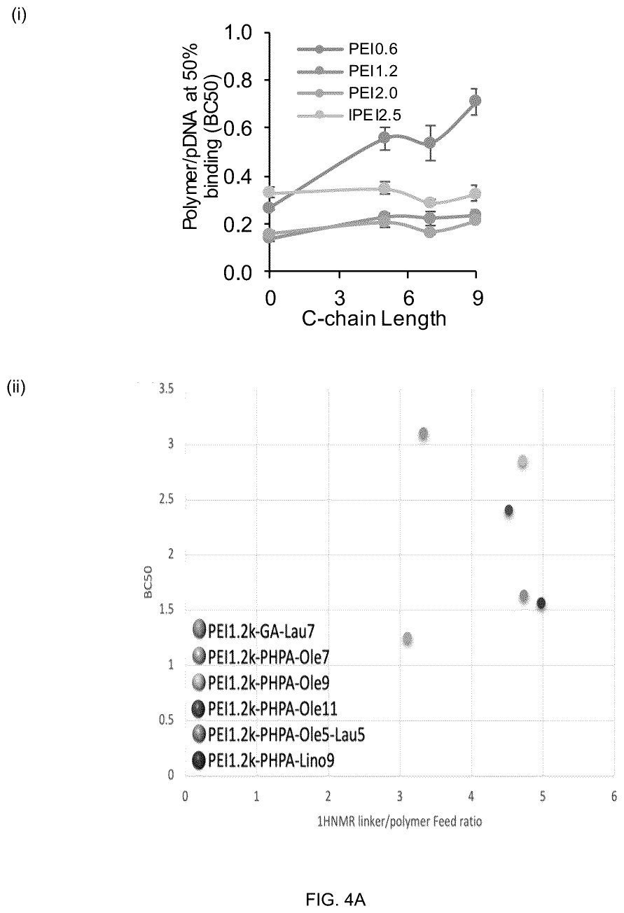

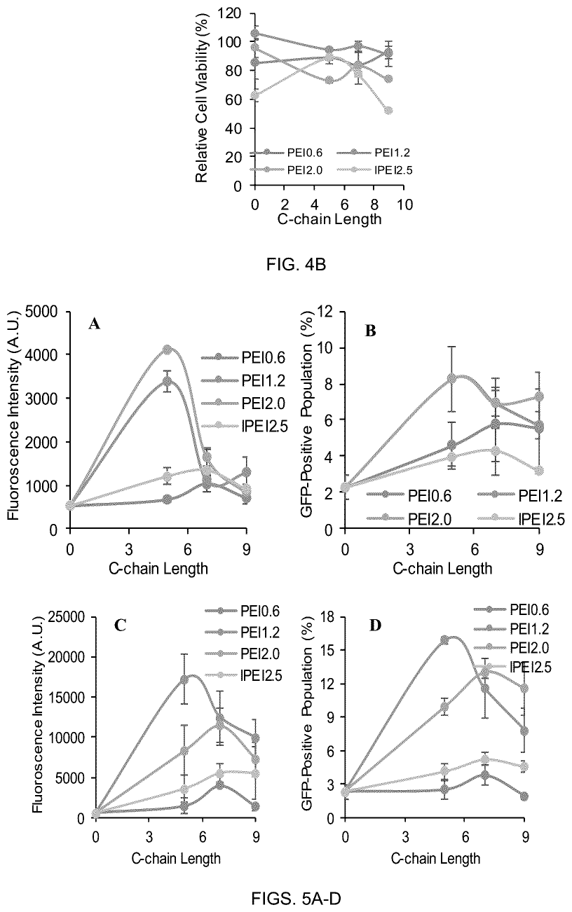

To investigate DNA binding capacity as a result of crosslinking, pDNA binding efficiency was evaluated by measuring the amount of free pDNA remaining after complex formation using the dye-exclusion assay. The fluorescence intensity of dye-pDNA intercalated complex was linearly decreased with the polymer concentration indicating the formation of the polyplex. The binding capacity of the parent polymers (PEI0.6, PEI1.2, PEI2.0, PEI2.5), as determined by BC₅₀ (i.e., weight ratio required for 50% binding of siRNA) was in the range of 0.15 to 0.3, while it was increased up to 0.7 with PEI0.6-tt. The effect of crosslinking was not observed to be that much significant in other polymers (FIG. 4A(i)). The BC₅₀ value for the GA and PUPA modified lipopolymers was also higher than the native PEIs (FIG. 4A(ii)). In general, the binding capacity of PEIs was inversely proportional to the amount of lipid moieties substitution, where the higher substitution resulted in a higher BC₅₀ value. Contrary to this phenomenon, in crosslinked PEIs, the BC₅₀ value was nearly the same as that of the parent polymers except PEI0.6. Without being bound by any theory, the impact of lipid chain of crosslinking may have been insignificant over the subsequently added PEI units as the result of crosslinking.

### Toxicity of Polymer/DNA Complexes

The PEI-tt/DNA complexes exhibited minimal toxicity in Jurkat cells. Toxicity of the complexes was increased with C-chain length of the tt-linkers which was higher in the polymers of higher molecular weight (FIG. 4B). The polymers/DNA complexes of branched PEIs at optimum formulation (PEI1.2 and PEI2.0 at ratio 5.0, w/w and PEI0.6 at ratio 15.0, w/w) exhibited 10 to 20% cellular toxicity. The toxicity of lPEI-tt/DNA complexes (ratio 5.0, w/w) was 5 to 40% based on the tt-linkers.

DNA Transfection with PEI-Tt Library

A thioester crosslinked PEIs library was screened for transfection efficiency in Jurkat cells using gWIZ-GFP as a reporter gene and lPEI40 as a positive control. The screen was performed in a wide range of polymer/DNA ratios to determine the optimal composition for the most effective transfection efficiency. The outcome of polymers synthesized with amide bonding was insignificant (not shown) compared to thioester crosslinking indicating the relevance of specific covalent bonding (e.g., cleavable) along with molecular weight of the carriers. In the latter case, the effect of crosslinking was significantly higher indicating the beneficial effect of crosslinking though it was dependent on the molecular weight of the parent polymer. It was found that the higher the molecular weight, the better the efficiency (FIGS. 5A-D). The outcome based on topology (branch vs linear) was not significant. Without being bound by any theory, the enhanced transfection efficiency of thioester linked PEIs might be due to increase plasmid delivery efficiency due to higher MW and/or reductive cleavage of labile bonding [56, 57]. The leading polymers, PEI0.6-tt5, PEI0.6-tt7, PEI0.6-tt9 and PEI1.2-tt5, PEI1.2-tt7, PEI1.2-tt9 with the composition of 1:1 (PEI:tt) were selected for further evaluation. Fluorescence images of Jurkat cells after transfection with GFP exclusively validate the higher transfection efficiency of PEI-tt polymers (FIG. 8). The outcomes were further confirmed by the flowcytometry assay which showed 2 to 2.5-fold increased mean fluorescence intensity.

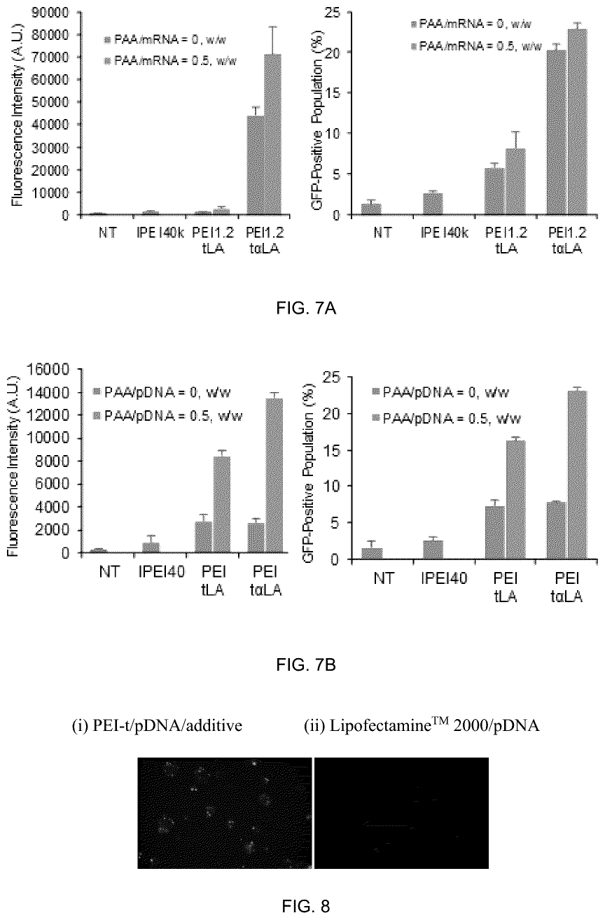

Transfection efficiency in Jurkat T-cells was studied using PEI-tLA, PEI-tαLA polymers along with commercial transfection reagents, Lipofectamine™ 2000, 25 kDa branched PEIs (PEI25) and 40 kDa linear PEI (lPEI40) (FIGS. 7A-B). In each case, transgene expression with PEI-tLA, PEI-tαLA significantly dominated over commercial reagent, indicating the beneficial effect of aliphatic lipid substitution in low molecular (e.g. 1.2 kDa) weight PEIs. The effect of longer (C14 to 22 with 1 to 3 unsaturation) lipids substitution onto PEI1.2 via thioester bonding was more beneficial for transfection of Jurkat cells. This indicates the relevance of proper lipids and proper chemical bonding (cleavage) for higher transfection in lymphoid cells. These two parameters are proven triggers for gene delivery in both in vitro and in vivo model due to better cellular uptake, stimuli assisted unpacking of the complexes for prompt release of the payload in the site of interest.

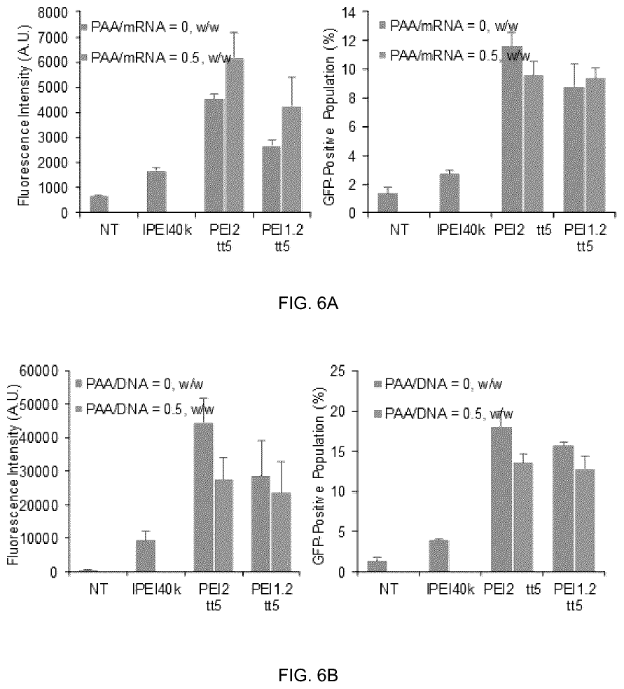

Polyanions were inserted into polymer/nucleic acid complexes as an additive to enhance transfection efficiency (FIGS. 6A-B, 7A-B). These insertions were beneficial for unpacking of the complexes due to competitive interaction of polyanions with the cationic polymers. The inventors have been exploring a wide range of polyanionic macromolecules such as polyacrylic acid, polyaspartic acid, hyaluronic acid, gelatin for these particular formulations. In general, at a proper balanced composition (e.g., Additive: DNA=0.2 to 2, w/w) these formulations showed significantly higher transfection efficiency in hard-to-transfect cells. In Jurkat cells, as an example, addition on polyacrylic acid into PEI-tαLA/DNA complexes showed 5 to 6-fold increase trans gene expression. The enhanced transfection efficiency was the cause of easy complexes unpacking.

Two broadly acting commercial transfection reagents (Lipofectamine™ 2000 and high molecular weight (25 kDa) PEI) were ineffective for DNA delivery in anchorage independent Jurkat cells with the delivery of gWIZ-GFP (FIG. 8). Without being bound by any theory, this clearly demonstrated the utility of crosslinking or hydrophobic modification of low molecular weight PEIs via thioester trigger for effective delivery polynucleotides.

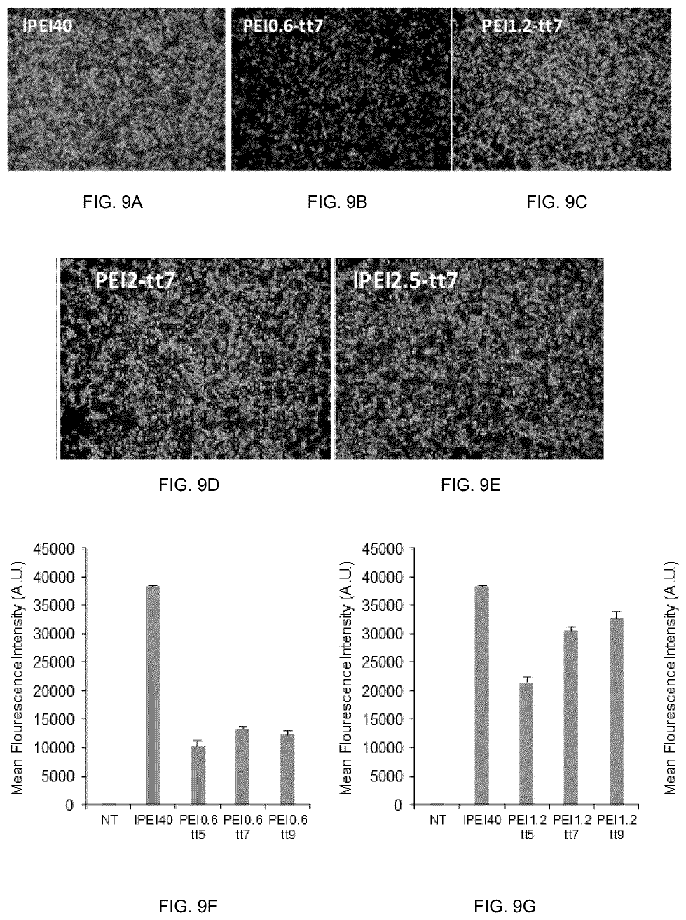

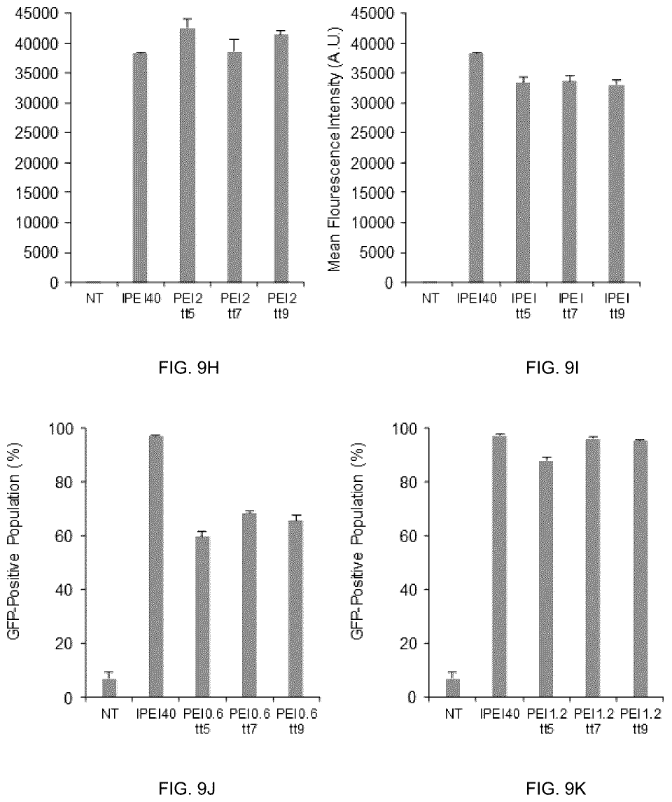

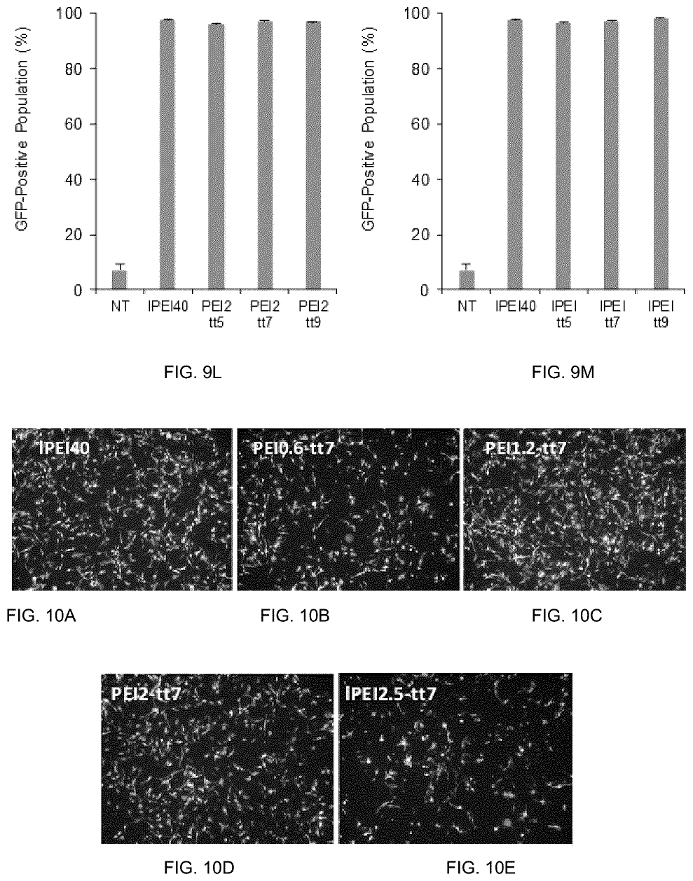

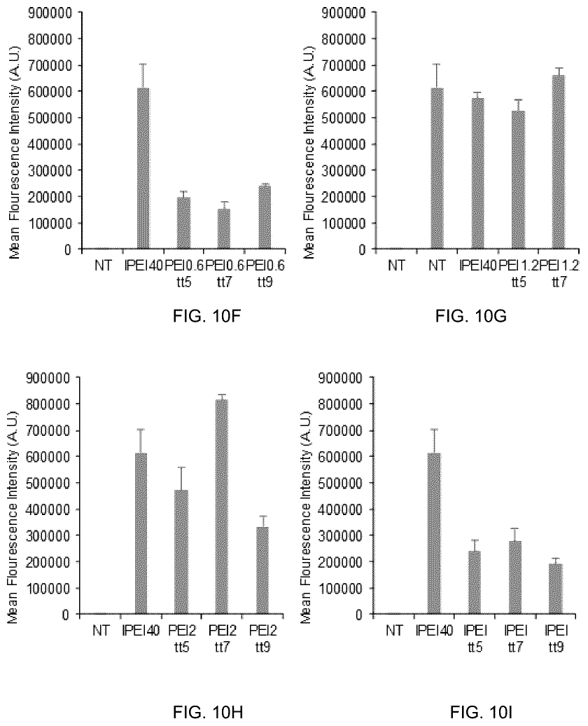

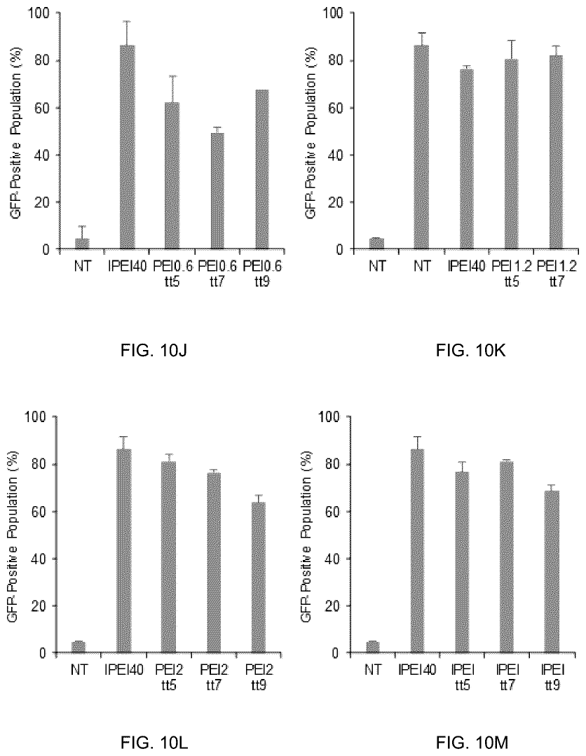

Transfection efficiency of PEI-tt polymers was also studied using human kidney epithelial 293T cells (FIGS. 9A-M) and breast cancer MDA-MB-436 cells (FIGS. 10A-M). In each case, transgene expression with PEI-tt polymers were equivalent or superior to commercial reagent lPEI40, indicating the utility of PEI-tt polymers in other types of cells.

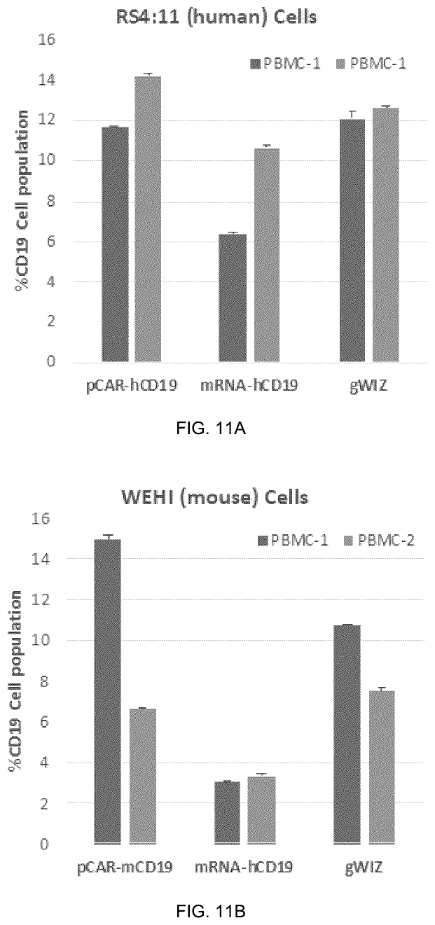

Cell killing activity of the modified T-cells was studied using human T-cells derived from blood. The PBMCs were isolated for this purpose and enriched for T-cells using TL-2 in the culture medium. FIGS. 11A-B shows cell killing by 2 sources of human PBMCs that were modified with a plasmid DNA and a mRNA expression system for CAR directed against human (FIG. 11A) and mouse (FIG. 11B) CD19. The polymer used in this study was PEI-tLA and complexes were formed with the polyanionic additives. In FIGS. 11A-B, where the modified cells were incubated with human CD19+RS4;11 and mouse CD19(+) WEHI cells, respectively, it can be seen that incubation with the mRNA-based expression system resulted in significant reduction of the CD19(+) cell population, confirming effective modification of the cells with the prepared polymers. While the inventors have previously reported the ability of PEI thioester-linked with a lipid (PEI-t) to deliver pDNA to cells from bone marrow and express transgene [53], this example demonstrates that they are more potent and effective to deliver mRNA to express therapeutic proteins.

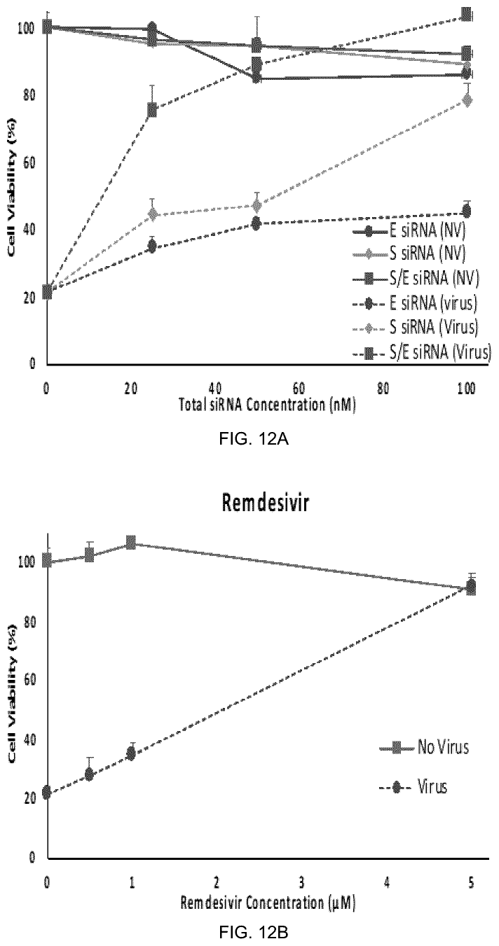

Transfection efficiency of PEI-tt polymers was studied using African green monkey epithelial Vero cells (FIGS. 12A-B). In these cells, the polymers were complexed with siRNAs specific against the coronavirus 229E, so as to stop the replication of the coronavirus in infected cells. Three separate siRNAs were used, targeting E, N and S proteins of the coronavirus 229E. The Vero cells were treated with nanoparticles composed of siRNAs and lipid-substituted polymers, after which they were exposed to coronavirus infection for 5 days. As can be seen in FIG. 12A, the cell survival in coronavirus infected cells was significantly improved with increasing concentration of siRNA delivery by using the lipid-substituted polymers, indicating halting of the coronavirus synthesis in the infected cells. The anti-viral drug remdesivir gave a similar response as nanoparticles composed of siRNA and the described polymers (FIG. 12B).

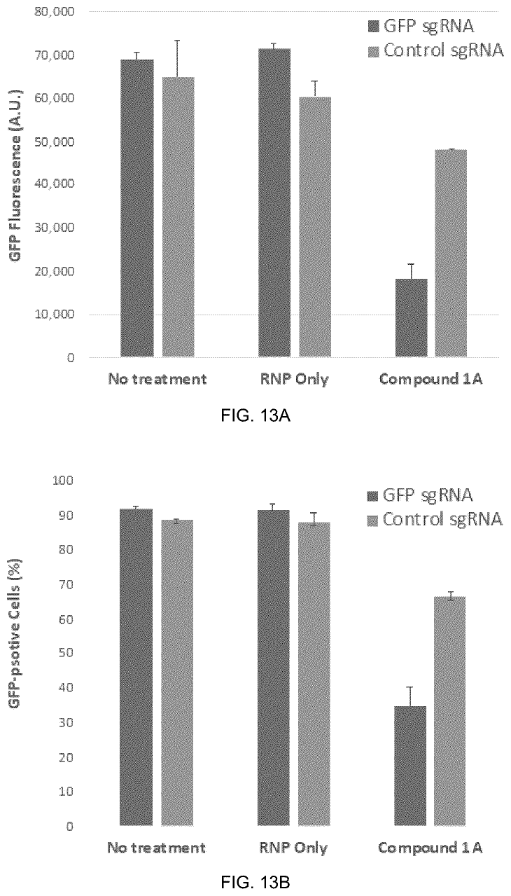

The ability of the polymers to edit host genome was also studied using Cas9 enzyme in MDA-MB-231 cells (FIGS. 13A-B) that are modified to express GFP transgene. The ribonucleic acid protein (RNP) complex was first formed by mixing sgRNA and Cas9 enzyme. The sgRNA were either non-specific (control) or specific against the GFP, so as to reduce the expression of GFP. As shown in FIGS. 13A-B, no changes in GFP expression were observed when cells were exposed to RNP alone without the transfection reagent, while GFP fluorescence (FIG. 13A) and percentage of GFP-positive cells (FIG. 13B) were readily reduced by treatment of the cells with complexes composed of GFP sgRNA specific RNPs and lipid-substituted polymers.

Antitumor Activity of TRAIL mRNA in a Xenograft Model

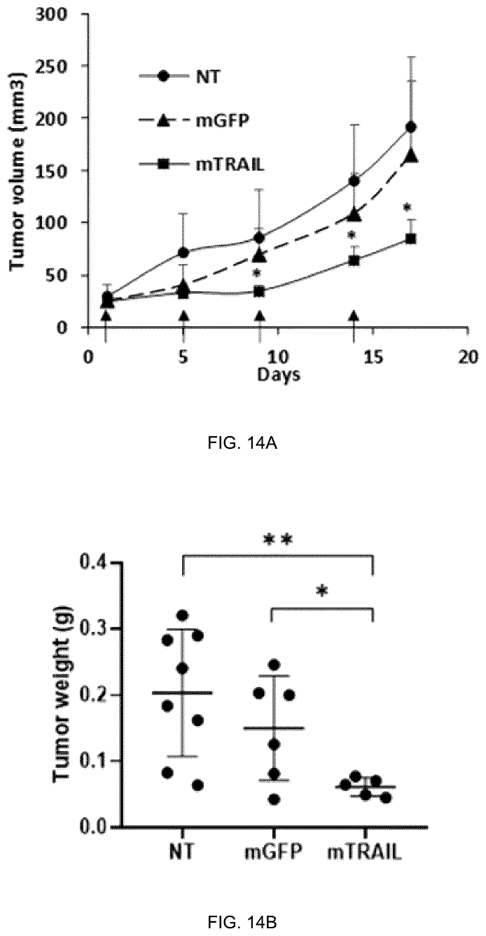

The effectiveness of the Polymer IA to deliver TRAIL mRNA was evaluated using SUM-149 xenografts (FIG. 14A) in mice. TRAIL is a protein that selectively inhibits the growth of breast cancers cells such as SUM-149 cells in vitro. The growth of the tumors in the mGFP and the no-treatment group were not significantly different, indicating no effect of GFP coding mRNA on tumor growth (as expected). Treatment with TRAIL mRNA significantly reduced the tumor volume after day 9. The tumor weights recovered at the end of the study were in line with the external tumor volumes measured, where the lowest mean tumor weight was observed with the mTRAIL treatment formulated with Polymer IA (FIG. 14B).

It should be apparent, however, to those skilled in the art that many more modifications besides those already described are possible without departing from the inventive concepts herein. The inventive subject matter, therefore, is not to be restricted except in the scope of the disclosure. In interpreting the disclosure, all terms should be interpreted in the broadest possible manner consistent with the context. The terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

### REFERENCES

1. M. F. Lorenzo, S. C. Thomas, Y. Kani, J. Hinckley, M. Lee, J. Adler, S. S. Verbridge, F. C. Hsu, J. L. Robertson, R. V. Davalos, J. H. Rossmeisl, Jr., Temporal Characterization of Blood-Brain Barrier Disruption with High-Frequency Electroporation, Cancers (Basel), 11 (2019).
2. N. Bono, F. Ponti, D. Mantovani, G. Candiani, Non-Viral in Vitro Gene Delivery: It is Now Time to Set the Bar!, Pharmaceutics, 12 (2020).
3. U. Lachelt, E. Wagner, Nucleic Acid Therapeutics Using Polyplexes: A Journey of 50 Years (and Beyond), Chem Rev, 115 (2015) 11043-11078.
4. B. R. Olden, Y. Cheng, J. L. Yu, S. H. Pun, Cationic polymers for non-viral gene delivery to human T cells, J Control Release, 282 (2018) 140-147.
5. Park, J. H., and Renier J. B. (2010). Adoptive immunotherapy for B-cell malignancies with autologous chimeric antigen receptor modified tumor targeted T cells”. Dis. Medicine 9, 277-288.
6. Jorgensen, J. L., Reay, P. A., Ehrich, E. W., Davis, M. M. (1992). Molecular components of T-cell recognition. Annu. Rev. Immunol. 10, 835-873.
7. Mitchison, N. A. (1955). Studies on the immunological response to foreign tumor transplants in the mouse. I The role of lymph node cells in conferring immunity by adoptive transfer. J Exp Med. 102, 157-177.
8. Rosenberg S A, Packard B. S, Aebersold P. M, Solomon D., Topalian S. L., Toy, S. T., Simon, P., Lotze, M. T., Yang J. C., Seipp, C. A. (1988) Use of tumor infiltrating lymphocytes and interleukin-2 in the immunotherapy of patients with metastatic melanoma. A preliminary report. N. Engl. J. Med. 319, 1676-1680
9. Riddell, S. R., Watanabe, K. S., Goodrich, J. M., Li, C. R., Agha, M. E., Greenberg, P. D. (1992). Restoration of viral immunity in immunodeficient humans by the adoptive transfer of T cell clones. Science 257, 238-241
10. Levine, B L., Bernstein, N. E., Aronson, K., Schlienger, J., Cotte, S., Perfetto, M. J., et al. (2002). Adoptive transfer of costimulated CD4+ T cells induces expansion of peripheral T cells and decreased CCR5 expression in HIV infection. Nat. Med. 8, 47-53
11. Sadelain, M., Riviere, I., Brentjens, R. (2003). Targeting tumours with genetically enhanced T lymphocytes. Nat. Rev. Cancer 3, 35-45.
12. Hicklin, D. J., Marincola, F. M., Ferrone, S. (1999). HLA class I antigen down regulation in human cancers: T-cell immunotherapy revives an old story. Mol. Med. Today 5, 178-86.
13. Khong, H. T., Restifo, N. P. (2002). Natural selection of tumor variants in the generation of “tumor escape” phenotypes. Nature Immunol. 3, 999-1005.
14. National Cancer Institute. (2017). CAR T cells: Engineering patients' immune cells to treat their cancers. Retrieved from https://www.cancer.gov/about-cancer/treatment/research/car-t-cells
15. Engel, P., Zhou, L. J., Ord, D. C., Sato, S., Koller, B., Tedder, T. F. (1995) Abnormal B lymphocyte development, activation, and differentiation in mice that lack or overexpress the CD19 signal transduction molecule. Immunity 3, 39-50.
16. Kymriah® (tisagenlecleucel), first-in-class CAR-T therapy from Novartis, receives second FDA approval to treat appropriate r/r patients with large B-cell lymphoma (2018). https://www.novartis.com/news/media-releases/kymriahr-tisagenlecleucel-first-class-car-t-therapy-from-novartis-receives-second-fda-approval-treat-appropriate-rr-patients-large-b-cell-lymphoma.
17. YESCARTA™ (axicabtagene ciloleucel) suspension for intravenous infusion Initial U. S. Approval: 2017. https://www.fda.gov/downloads/UCM581226.pdf
18. Katz, B. Z & Herishanu, Y. (2014). Therapeutic targeting of CD19 in hematological malignancies: past, present, future and beyond. Leuk. Lymph. 55, 999-1006.
19. Park, J. H., Geyer, M. B and Brentjens, RJ. (2016) CD19-targeted CAR T-cell therapeutics for hematologic malignancies: interpreting clinical outcomes to date. Blood 127, 3312-3320.
20. Brentjens R. J. (2009). Cellular therapies in acute lymphocytic leukemia. Curr. Opin. Mol. Ther. 11, 375-382.
21. Savoldo, B., Ramos, C. A., Liu, E., Mims, M. P., Keating, M. J., Carrum, G., et al. (2011). CD28 costimulation improves expansion and persistence of chimeric antigen receptor-modified T cells in lymphoma patients. J. Clin. Invest. 121, 1822-6.
22. Maude, S. L., Frey, N., Shaw, P. A., Aplenc, R., Barrett, D. M., Bunin, N. J., et al. (2014). Chimeric antigen receptor T cells for sustained remissions in leukemia. N Engl J Med 371, 1507-1517.
23. Kalos, M., Levine, B. L., Porter, D. L., Katz, S., Grupp, S. A., Bagg, A., and June, C. H. (2011). T cells with chimeric antigen receptors have potent antitumor effects and can establish memory in patients with advanced leukemia. Sci. Transl Med, 3, 95ra73.
24. Kawalekar, O. U., O'Connor, R. S., Fraietta, J. A., Guo, L., McGettigan, S. E., Posey, A. D., et al., (2016) Distinct signaling of coreceptors regulates specific metabolism pathways and impacts memory development in CAR T cells. Immunity 44, 380-390.
25. van der Stegen, S. J., Hamieh, M., and Sadelain, M. (2015) The pharmacology of second-generation chimeric antigen receptors. Nature Rev. Drug Disco. 14, 499-509.
26. Jiang B, Yan L, Wang X, Li E, Murphy K, Vaccaro K, Li Y, Xu R H. (2019) Mesenchymal Stem Cells Derived from Human Pluripotent Cells, an Unlimited and Quality-Controllable Source for Therapeutic Applications. Stem Cells. 37, 572-581.
27. Zhang, C., Liu, J., Zhong, J. F., and Zhang, X. (2017). Engineering CAR-T cells. Biomarker Research, 5, 22.
28. Hacein-Bey-Abina, S., Garrigue, A., Wang, G. P., Soulier, J., Lim, A., Morillon, E., et al. (2008). Insertional oncogenesis in 4 patients after retrovirus-mediated gene therapy of SCID-X1. J. Clin. Invest. 118, 3132-42.
29. Wang, G. P., Garrigue, A., Ciuffi, A., Ronen, K., Leipzig, J., Berry, C., et al. (2008). DNA bar coding and pyrosequencing to analyze adverse events in therapeutic gene transfer. Nucleic Acids Res. 36, e49.
30. Wang, X., Olszewska, M., Qu, J., Wasielewska, T., Bartido, S., Hermetet, G. et al. (2015). Large-scale clinical-grade retroviral vector production in a fixed-bed bioreactor. J. Immunother. 38, 127-35.
31. Przybylowski, M., Hakakha, A., Stefanski, J., Hodges, J., Sadelain, M., and Riviere, I. (2005). Production scale-up and validation of packaging cell clearance of clinical-grade retroviral vector stocks produced in cell factories. Gene Therapy, 13, 95.
32. Levine, B. L., Miskin, J., Wonnacott, K., and Keir, C. (2016). Global Manufacturing of CAR T Cell Therapy. Mol. Ther. Meth. Clin. Develop, 4, 92-101.
33. Kebriaei, P., Izsvik, Z., Narayanavari, S. A., Singh, H., and Ivics, Z. (2017). Gene therapy with the sleeping beauty transposon system. Tren. Genet. 33, 852-870.
34. Ivics, Z., Li, M. A., Mites, L., Boeke, J. D., Nagy, A., Bradley, A., and Izsvik, Z. (2009). Transposon-mediated genome manipulation in vertebrates. Nature Methods 6, 415-22.
35. Wells, D. J. (2004). Gene therapy progress and prospects: Electroporation and other physical methods. Gene Therapy, 11, 1363.
36. Holstein, M., Mesa-Nunez, C., Miskey, C., Almarza, E., Poletti, V., Schmeer, M., et al. (2018). Efficient non-viral gene delivery into human hematopoietic stem cells by minicircle Sleeping Beauty transposon vectors. Mol. Ther. 26, 1137-1153.
37. Zhou, Z., Liu, X., Zhu, D., Wang, Y., Zhang, Z., Zhou, X., et al. (2017). Nonviral cancer gene therapy: Delivery cascade and vector nanoproperty integration. Adv. Drug Del. Rev. 115, 115-154.
38. Moon, J. J., Suh, H., Bershteyn, A., Stephan, M. T., Liu, H., Huang, B., et al. (2011). Interbilayer-crosslinked multilamellar vesicles as synthetic vaccines for potent humoral and cellular immune responses. Nature Mat. 10, 243-51.
39. Olden, B. R., Cheng, Y., Yu, J. L., and Pun, S. H. (2018). Cationic polymers for non-viral gene delivery to human T cells. J. Contr. Rel. 282, 140-147.
40. Smith, T. T., Stephan, S. B., Moffett, H. F., McKnight, L. E., Ji, W., Reiman, D., et al. (2017) In situ programming of leukaemia-specific T cells using synthetic DNA nanocarriers. Nature Nanotech. 12, 813-820.
41. C. M. Wells, M. Harris, L. Choi, V. P. Murali, F. D. Guerra, J. A. Jennings, Stimuli-Responsive Drug Release from Smart Polymers, J Funct Biomater, 10 (2019).
42. F. Q. Schafer, G. R. Buettner, Redox environment of the cell as viewed through the redox state of the glutathione disulfide/glutathione couple, Free Radic Biol Med, 30 (2001) 1191-1212.
43. M. Breunig, U. Lungwitz, R. Liebl, A. Goepferich, Breaking up the correlation between efficacy and toxicity for nonviral gene delivery, Proc Natl Acad Sci USA, 104 (2007) 14454-14459.
44. P. A. Fernandes, M. J. Ramos, Theoretical insights into the mechanism for thiol/disulfide exchange, Chemistry, 10 (2004) 257-266.
45. P. J. Bracher, P. W. Snyder, B. R. Bohall, G. M. Whitesides, The relative rates of thiol-thioester exchange and hydrolysis for alkyl and aryl thioalkanoates in water, Orig Life Evol Biosph, 41 (2011) 399-412.
46. Zintchenko A, Philipp A, Dehshahri A, Wagner E. Simple modifications of branched PEI lead to highly efficient siRNA carriers with low toxicity. Bioconjugate chemistry. 2008; 19:1448-55.;
47. Lungwitz U, Breunig M, Blunk T, Gopferich A. Polyethylenimine-based non-viral gene delivery systems. European journal of pharmaceutics and biopharmaceutics: official journal of Arbeitsgemeinschaft fur Pharmazeutische Verfahrenstechnik eV. 2005; 60:247-66.;
48. Aigner A, Fischer D, Merdan T, Brus C, Kissel T, Czubayko F. Delivery of unmodified bioactive ribozymes by an RNA-stabilizing polyethylenimine (LMW-PEI) efficiently down-regulates gene expression. Gene therapy. 2002; 9:1700-7.
49. Wei Dong, Yunzhen Wei. A biodegradable crosslinked polyethyleneimine and its uses. International Publication No. WO 2008/058457 A1, published May 22, 2008.
50. Yasunobu Tanaka, Gang Zhao, Lei Yu. Biodegradable cationic polymers. U.S. Pat. No. 7,700,541 B2, issued Apr. 20, 2010.
51. Zhijun, Z. et al. Degradable gene vector based on polyethyleneimine and preparation method thereof. Chinese Patent Application No. CN 103509183A, published Jan. 15, 2014.
52. Sheng Li, Chris Castello, Sang Van. Cationic polymers having degradable crosslinks. United States Patent Application Publication No. 2005/0089503 A1, published Apr. 28, 2005.
53. Remant Bahadur K. C., C. Kucharski, H. Uludag. Additive nanocomplexes of cationic lipopolymers for improved non-viral gene delivery to mesenchymal stem cells. J. Mat. Chem. B (2015) 3: 3972-3982.
54. A. Neamnark, O. Suwantong, R. K. Bahadur, C. Y. Hsu, P. Supaphol, H. Uludag, Aliphatic lipid substitution on 2 kDa polyethylenimine improves plasmid delivery and transgene expression, Mol Pharm, 6 (2009) 1798-1815.
55. J. P. Nam, S. Kim, S. W. Kim, Design of PEI-conjugated bio-reducible polymer for efficient gene delivery, Int J Pharm, 545 (2018) 295-305.
56. H. S. Hwang, H. C. Kang, Y. H. Bae, Bioreducible polymers as a determining factor for polyplex decomplexation rate and transfection, Biomacromolecules, 14 (2013) 548-556.
57. M. Breunig, U. Lungwitz, R. Liebl, C. Fontanari, J. Klar, A. Kurtz, T. Blunk, A. Goepferich, Gene delivery with low molecular weight linear polyethylenimines, J Gene Med, 7 (2005) 1287-1298.

1. A compound comprising a polymer having a molecular weight ranging from about 0.5 kDa to about 5 kDa and an aliphatic lipid-thioester group,
wherein the polymer is selected from polyethylenimine in a branched, linear, or dendritic form, polyalkylimine, a poly(amino acid), a poly(beta-amino acid), a poly(beta-amino ester), a cationic amino acid containing a peptide or a polymer, an aminated polymer derived from water-soluble, uncharged polymers modified with amine compounds, polyethylenimine derivatized with silica, polyethylenglycol, polypropyleneglycol, an amino acid, dopamine, poly(2-dimethylaminoethyl methacrylate or a derivative thereof in combination with a polymer to create amphiphilic polymers; a polyamidoamine derivative; and poly(N-(2-hydroxypropyl)methacrylamide) or a derivative thereof, and
wherein the aliphatic lipid comprises a saturated or unsaturated aliphatic lipid selected from propanoyl (C3), propanedioyl (C3), pentanedioyl or glutaryl chloride (C5), hexanoic acid or hexanoyl (C6), heptanedioyl or pimeloyl chloride (C7), capryloyl (C8), lipoic acid or lipoyl (C8), nonanedioyl or azelaoyl chloride (C9), lauric acid or lauroyl (C12), dodecanedioyl (C12), palmitic acid or palmitoyl (C16), stearoyl (C18), linoleoyl (C18), oleoyl (C18), eicosanoyl (C20), eicosapentaenoyl (C20), arachidonoyl (C20), linolarachidonoyl (C20), docosanoyl (22), docosahexaenoyl (22), myristoleic acid (C14:1, cis-9), palmitoleic acid (C16:1, cis-9), stearic acid (C18) and derivatives thereof, oleic acid (C18:1, cis-9), elaidic acid (C18:1, trans-9), linoleic acid (C18:2, cis-9,12), or linolenic acid (C18:3, cis-9,12,15).

2. The compound of claim 1, wherein the polymer comprises polyethylenimine, and the aliphatic lipid-thioester group has the formula IIIA or IIIB: 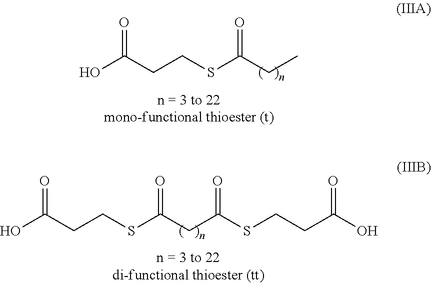
where n is the carbon chain length ranging from C3 to C22.

3. The compound of claim 2, having the formula IA: 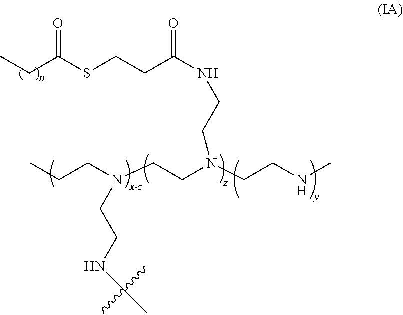
wherein the aliphatic lipid-thioester group has the formula IIIA, and a hydrophobic group comprises 3<n<22 atoms; x=5<n<30; y=5<n<30; and z=1<n<5.

4. The compound of claim 2, having the formula IB: 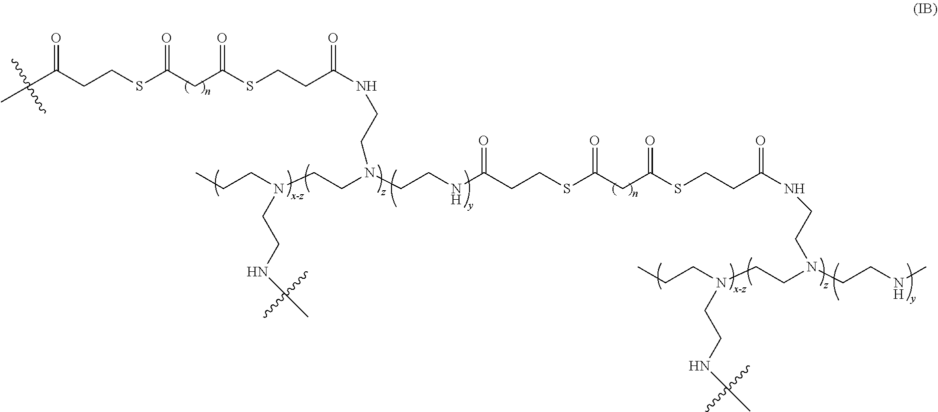
wherein the aliphatic lipid-thioester group has the formula IIIB, and a linker comprises a spacer having 3<n<22 atoms; x=5<n<30; y=5<n<30; and z=1<n<5.

5. A compound comprising a polymer having a molecular weight ranging from about 0.5 kDa to about 5 kDa and a lipid-ester or lipid-thioester group,
wherein the polymer is selected from polyethylenimine in a branched, linear, or dendritic form, polyalkylimine, a poly(amino acid), a poly(beta-amino acid), a poly(beta-amino ester), a cationic amino acid containing a peptide or a polymer, an aminated polymer derived from water-soluble, uncharged polymers modified with amine compounds, polyethylenimine derivatized with silica, polyethylenglycol, polypropyleneglycol, an amino acid, dopamine, poly(2-dimethylaminoethyl methacrylate or a derivative thereof in combination with a polymer to create amphiphilic polymers; a polyamidoamine derivative; and poly(N-(2-hydroxypropyl)methacrylamide) or a derivative thereof, and
wherein the lipid comprises a saturated or unsaturated aliphatic lipid selected from propanoyl (C3), propanedioyl (C3), pentanedioyl or glutaryl chloride (C5), hexanoic acid or hexanoyl (C6), heptanedioyl or pimeloyl chloride (C7), capryloyl (C8), lipoic acid or lipoyl (C8), nonanedioyl or azelaoyl chloride (C9), lauric acid or lauroyl (C12), dodecanedioyl (C12), palmitic acid or palmitoyl (C16), stearoyl (C18), linoleoyl (C18), oleoyl (C18), eicosanoyl (C20), eicosapentaenoyl (C20), arachidonoyl (C20), linolarachidonoyl (C20), docosanoyl (22), docosahexaenoyl (22), myristoleic acid (C14:1, cis-9), palmitoleic acid (C16:1, cis-9), stearic acid (C18) and derivatives thereof, oleic acid (C18:1, cis-9), elaidic acid (C18:1, trans-9), linoleic acid (C18:2, cis-9,12), or linolenic acid (C18:3, cis-9,12,15); triglyceride including glyceryl tridecanoate, glyceryl tridodecanoate, glyceryl trimyristate, glyceryl trioctanoate, tripalmitin; lipoic acid and derivatives thereof in oxidized and reduced form; cholesterol and derivatives thereof including cholic acid, deoxycholic acid, and cholanic acid; phospholipid selected from α-phosphatidylcholine, α-phosphatidylethanolamine, α-phosphatidyl-L-serine, α-phosphatidylinositol, α-phosphatidic acid, α-phosphatidyl-DL-glycerol, α-lysophosphatidylcholine, sphingomyelin, cardiolipin; synthetic lipidic compounds including diphytanoyl phosphatidylethanolamine (DPHPE), dioleoyl phosphatidylethanolamine (DOPE), dioleoyl phosphatidylcholine (DOPC), dilauryl phosphatidylethanolamine (DLPE), 1,2-distearoyl-sn-glycero-3-phosphatidylethanolamine (DSPE), and dimyristoyl phosphatidylethanolamine (DPME); multicyclic lipids and steroids including cholesterol, cholestanol, coprosterol, epicholestanol, epicholesterol, ergostanol, [alpha]-ergostenol, [beta]-ergostenol, [gamma]-ergostenol, ergosterol, 22,23-dihydroergosterol, stigmasterol, stigmastanol, (3[beta])-7-dehydrocholesterol, desmosterol, allocholesterol, 24-hydroxycholesterol, 25-hydroxycholesterol, campesterol, [alpha]-sitosterol, [beta]-sitosterol, [gamma]-sitosterol, lumisterol, pyrocalciferol, isopyrocalciferol, azacosterol, neoergosterol, and dehydroergosterol.

6. The compound of claim 5, wherein the polymer comprises polyethylenimine, and the lipid-ester or lipid-thioester group has the formula selected from formula IVA, IVB, IVC, or IVD: 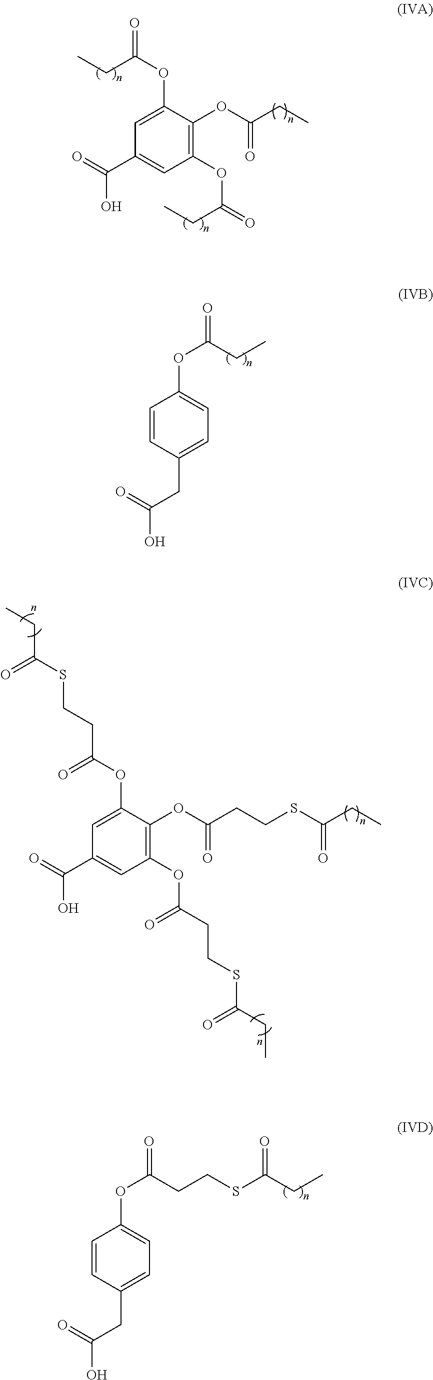
where n is the carbon chain length ranging from C3 to C22.

7. The compound of claim 6, having the formula IIA, IIB, IIC, or IID: 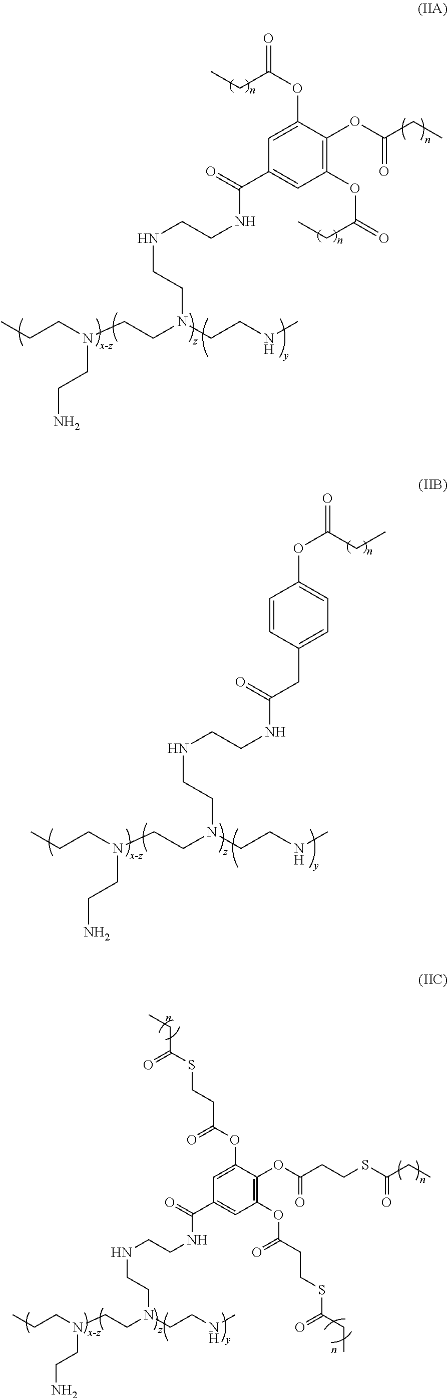 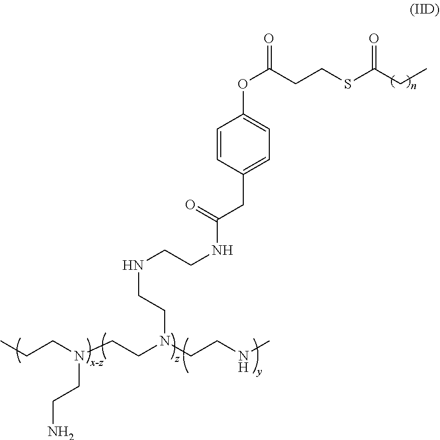
where the compound comprises a carbon chain length of 3<n<22 atoms; x=5<n<30; y=5<n<30; and z=1<n<5.

8. A nanoparticle comprising the compound of claim 3 complexed to a nucleic acid, or a composition comprising the nanoparticle or the compound, and a pharmaceutically acceptable carrier.

9. The nanoparticle of claim 8, wherein the nucleic acid is selected from an RNA-based nucleic acid comprising siRNA, sgRNA, microRNA, mRNA, shRNA, or combinations thereof, a DNA-based nucleic acid comprising a DNA-based oligonucleotide or antisense oligonucleotide, plasmid DNA for encoding an RNA product comprising shRNA, mRNA, sgRNA, or combinations thereof, a peptide-nucleic acid; a DNA-RNA chimera; or a nucleic acid in combination with a protein.

10. The nanoparticle of claim 9, further comprising an additive selected from polyanions, polyacrylic acid, polymethacrylic acid, polyaspartic acid, polyglutamic acid, gelatin, hyaluronic acid, cellulose, or derivatives thereof.

11. (canceled)

12. A method of treating, preventing, or ameliorating a disease in a subject, comprising administering to the subject an effective amount of a nanoparticle comprising the compound of claim 3 complexed to a nucleic acid; or a composition comprising the compound or the nanoparticle, and a pharmaceutically acceptable carrier.

13. The method of claim 12, wherein the disease comprises chronic and acute myeloid leukemia, chronic and acute lymphocytic leukemia, hairy cell leukemia, meningeal leukemia, myeloma, multiple myeloma, lymphoma, brain cancer, bladder cancer, breast cancer, melanoma, skin cancer, epidermal carcinoma, colon and rectal cancer, lung cancer, non-Hodgkin lymphoma, Hodgkin lymphoma, Sezary Syndrome, endometrial cancer, pancreatic cancer, kidney cancer, prostate cancer, leukemia thyroid cancer, head and neck cancer, ovarian cancer, hepatocellular cancer, cervical cancer, sarcoma, gastric cancer, gastrointestinal cancer, and uterine cancer.

14. The method of claim 12, wherein the disease is treated, prevented, or ameliorated in the subject through genetically modified hematopoietic host cells.

15. The method of claim 14, wherein the host cells are selected from T-cells including helper T-cells, or Natural Killer cells.

16. The method of claim 12, wherein the nanoparticle or the composition comprises a nucleic acid selected from DNA or RNA.

17. (canceled)

18. A method of delivering mRNA or RNP complexes using the compound of claim 3.

19. (canceled)

20. (canceled)

21. A nanoparticle comprising the compound of claim 4 complexed to a nucleic acid, or a composition comprising the nanoparticle or the compound, and a pharmaceutically acceptable carrier.

22. A nanoparticle comprising the compound of claim 7 complexed to a nucleic acid, or a composition comprising the nanoparticle or the compound, and a pharmaceutically acceptable carrier.

23. A method of treating, preventing, or ameliorating a disease in a subject, comprising administering to the subject an effective amount of a nanoparticle comprising the compound of claim 4 complexed to a nucleic acid; or a composition comprising the compound or the nanoparticle, and a pharmaceutically acceptable carrier.

24. A method of treating, preventing, or ameliorating a disease in a subject, comprising administering to the subject an effective amount of a nanoparticle comprising the compound of claim 7 complexed to a nucleic acid; or a composition comprising the compound or the nanoparticle, and a pharmaceutically acceptable carrier.

25. A method of delivering mRNA or RNP complexes using the compound of claim 4.

26. A method of delivering mRNA or RNP complexes using the compound of claim 7.
